(19) United States
(12) Patent Application Publication
(10) Pub. No.: US 2020/0215085 A1
(43) Pub. Date: Jul. 9, 2020

(54) Method to Abate Acute Airway Hypersensitivity and Asthma Attacks

(71) Applicant: UTI Limited Partnership, Calgary (CA)

(72) Inventors: Richard J. A. WILSON, Calgary (CA); Nicholas JENDZJOWSKY, Calgary (CA); Arijit ROY, Calgary (CA)

(21) Appl. No.: 16/631,333

(22) PCT Filed: Jul. 19, 2018
(86) PCT No.: PCT/CA2018/000145

§ 371 Date: Jan. 15, 2020

Related U.S. Application Data
(60) Provisional application No. 62/534,638, filed on Jul. 19, 2017.

(51) Int. Cl. A61K 31/662; A61K 45/06; A61K 9/00; A61K 31/42; A61K 31/357; A61P 11/06; C07F 9/10

(52) U.S. Cl. CPC A61K 31/662; A61K 45/06; A61K 9/0019; C07F 9/10; A61K 9/0073; A61K 31/357; A61P 11/06; A61K 31/42

(57) ABSTRACT

Methods for prevention and treatment of asthma attacks involve the administration of one or more TRPV1 antagonists, one or more LPAr antagonists or preferably a combination of one or more TRPV1 antagonists and one or more LPAr antagonists. TRPV1 antagonists and/or LPAr antagonists or a combination of both inhibit or prevent carotid body activation during an acute asthma attack. TRPV1 antagonists, LPAr antagonists or a combination thereof are useful for preventing or ameliorating the symptoms of asthma attacks. Pharmaceutical compositions for use in treating asthma and more specifically for preventing or treating asthma attacks comprise a combination of a TRPV1 antagonist and an LPAr antagonist. Methods for making medicaments for such treatment are provided. Also provided are kits for treating asthma and for preventing or treating asthma attacks in which a TRPV1 antagonist and an LPAr antagonist are separately formulated for administration at the same time.

### BACKGROUND

Asthma is one of the most common lung diseases, affecting 241 million people worldwide and is the cause of 380,00 deaths per year¹. In the United States, on the order of 8% of the population has asthma and in 2014 over 3600 deaths were attributed to asthma².

Asthma is a chronic lung disease with inflammation and narrowing of the airways. Asthma is characterized by airflow limitation caused by inflammation, excess mucous secretion, remodelling changes, such as goblet cell metaplasia and increased smooth muscle mass and acute conducting airway constriction in response to stimuli, usually allergens (i.e., bronchoconstriction)³.

Asthma cannot be cured, but can in most cases be managed by at least avoiding triggers and use of inhaled short-acting β₂-agonists, corticosteroids and/or ipratroprium bromide, but inhaled drugs have only limited value in situations of severe bronchoconstriction and mucus plugging, when access to their site of action is blocked.

Asthma attacks (acute asthma or acute exacerbation of asthma) are however not uncommon and may require emergency room treatment with administration of inhaled or systemic medication and possibly oxygen. Asthma attacks may result from poorly controlled asthma. Poor control may result from inadequate treatment, failure to adhere to treatment, or the presence of other diseases or disorders (e.g., obesity). A subset of asthma patients have refractory or difficult to control asthma³, which does not respond to standard treatment noted above. Asthma attacks may develop gradually over a few days or may be sudden-onset due exposure to an allergen (trigger). Acute severe asthma is an acute exacerbation of asthma which does not respond to bronchodilators or steroids and as such is potentially life threatening. In acute severe asthma, a patient can exhibit severe breathlessness that makes it impossible to speak. The severity of such asthma attacks can be assessed, for example, by assessment of elevation of respiration and/or heart rate, decreased peak expiratory flow and/or reduced oxygen saturation. Additional levels of severity of asthma attacks include life-threatening asthma and near-fatal asthma⁴. Acute bronchoconstriction remains the leading cause of hospitalization and asthmatic sudden death, dictating the need for new medications.^{5,6,7} Understanding of how allergens trigger asthma is advancing rapidly⁷, but how immune responses cause bronchoconstriction remains an important knowledge gap.

In view of the high and increasing prevalence of asthma in the population and the significant potential for emergency intervention, particularly for acute severe asthma, there is a significant need in the art for additional and improved therapies for prevention and treatment of this disease and acute asthma attacks.

Asthma is typically characterized as inflammatory-mediated and corticosteroid/β-agonist responsive^{8,9,10}. However, asthmatic sufferers can display a diverse range of etiologies associated with Th2 specific asthma, IgE prevalent asthma and corticosteroid unresponsive asthma^{11,12,13,14}. Persistent asthma is associated with higher risk of cardiovascular disease¹⁵. The diverse phenotypes of asthma increase complexity of care leading to inadequate therapy which, in some cases, result in death⁷. Thus, there is a need in the art for therapies for the prevention and treatment of asthma, particularly acute asthma attacks and severe acute asthma attacks, that are effective for the various phenotypes of asthma.

This disclosure focuses on neuronal-regulated mechanisms of airway calibre¹⁶, which are common to all asthma phenotypes, for prevention and treatment options for asthma.

All lungs demonstrate acute airway bronchoconstriction in response to irritants such as capsaicin, but only asthmatic lungs respond acutely to allergens and/or bradykinin^{17,18,19,20,21,22,23,24}. The effects of allergens/bradykinin on asthmatic lungs is not simply the release of local inflammatory mediators activating strictly local reflexes^{16,17-24,25}, but likely involves a circuit that includes the brainstem because vagotomy annuls allergen-induced bronchoconstriction in animal models^{25, 26}. This circuit may be the lung vagal afferent to parasympathetic efferent reflex pathway that mediates the effects of irritants in naïve lungs^{16,25,26} or, as carotid body activation elicits bronchoconstriction, the afferent arm of this reflex may originate at the carotid body^{27,28,29,30,31,32,33,34,35,36,37}.

The carotid bodies consist of glomus cells, glia-like sustentacular cells and sensory afferents fibres^{38,39}. Carotoid bodies are sensory organs that detect changes in arterial blood oxygen which are located near the bifurcation of the carotid artery in the throat. The carotid bodies are reported to respond to hypoxia, hypercapnia, temperature, endocannabinoids and cytokines^{38,40,41,42}. Chemo-afferents from the carotid body project via the carotid sinus nerve (CSN) and glossopharyngeal nerve to synapses in the brainstem²⁸. Via this pathway, the carotid bodies are reported to activate cardiorespiratory circuits including parasympathetic efferents that innervate the lung and cause airway (e.g. bronchial) constriction⁴³. This pathway is reported to be activated in asthma and to contribute to bronchial hyper-responsiveness. Bronchial hyper-responsiveness is reported to be normalized in response to carotid body denervation^{44, 46}.

A role for the carotid body in asthma is further suggested by surgical therapy of carotid body resection for asthmatics. Early (and now largely abandoned) attempts to use unilateral carotid body resection in over 5000 humans as a treatment for asthma are not supported by clinical trials^{46,47} and to date, there is no conclusive evidence for the therapeutic effects of the more risky, but possibly more efficacious bilateral resection in humans^{48,49}. Further hindering the acceptance for a role of the carotid body in asthma, no feasible mechanism has emerged linking enhanced activity to the asthmatic lung^{50,51,52}.

A growing realization of the importance of the carotid bodies in sleep apnea and cardiovascular diseases has led to a resurgence of interest in their properties^{53,54,56}. Continuous positive airway pressure (CPAP) which raises measured oxygen in blood (PaO₂)^{56,57} and reduces carotid body activity is reported to diminish airway reactivity by 30%^{58,59,60,61}. These reports suggest a role for carotid body-regulated parasympathetic outflow in exacerbating asthmatic attacks⁶².

It has recently been reported that the exquisite heat sensitivity of the carotid body is mediated in large part by transient receptor potential cation channel vanilloid 1 receptors (TRPV1) in axons of chemosensory afferents (cell bodies in the petrosal ganglia, post-synaptic to oxygen-sensing glomus cella⁴¹). However, the physiological significance of this observation was unknown.

TRPV1 (Transient receptor potential cation channel subfamily V member 1) is a multimodal sensor capable of responding to heat, pH, anandamide and inflammatory mediators including IL-1⁶³. TRPV1 is also known as the capsaicin receptor or the vanilloid receptor. TRPV1 is reported to be genetically associated with childhood asthma^{64,86}. Activation of TRPV1 is reported to play a role in airway hypersensitivity which is associated with patients having airway inflammatory diseases, such as asthma⁶⁶. Ablation of neurons that are found in the vagal ganglia and characterized by the expression of TRPV1 ion channel are reported to abolish hyperreactive bronchoconstrictions even in the presence of a full lung inflammatory response. The expression of TRPV1 is reported to be increased by inflammatory stimuli⁶⁷. The expression of TRPV1 is reported to be increased in asthma patients⁶⁸ and in animal models of asthma⁶⁹. TRPV1 is reported to have a role in allergic asthma as a regulator of the activation and inflammatory properties of Ca₄⁺ cells⁷⁰.

Lysophosphatidic acid (LPA) is an endogenous and highly potent agonist of TRPV1 receptor^{71,72}. LPA is upregulated with oxidative stress and has recently been reported to be a major cause of asthmatic inflammation. LPA is produced by autotaxin (ATX)-mediated cleavage of lysophosphatidylcholine (LPC). Asthmatic lungs are reported to have an increased expression of ATX⁷³ and LPC^{74,75} and upon allergic provocation, LPA is reported to be readily produced and excreted into the blood.³³ In asthma, LPA blood concentrations are reported to range from <1-5 μM and to increase to 7-10 μM following asthmatic attacks^{76,77}.

LPA receptor antagonists have been proposed to be useful for the treatment of a disease or condition that would benefit from inhibition of the activity of at least one LPA receptor. See: U.S. Pat. No. 8,975,235, which is incorporated by reference herein in its entirety for disclosure therein of LPA receptor antagonists and uses thereof. LPA1 receptor antagonists have been proposed to be useful for the treatment of a variety of diseases including among others heart failure, cardiomyopathy, myocardial infarction, myocardial remodeling, vascular remodeling, hypertension, atherosclerosis, peripheral arterial occlusive disease (PAOD), restenosis, thrombosis, vascular permeability disorders, inflammation or inflammatory diseases such as rheumatoid arthritis, osteoarthritis, pulmonary diseases (such as chronic obstructive pulmonary disease, asthma or acute respiratory distress syndrome), immunological diseases, allergic diseases, tumor growth, metastasis, metabolic diseases, fibrotic diseases, collagenosis, scleroderma, progressive systemic sclerosis and nephrogenic fibrosing dermopathy, psoriasis, pain such as neuropathic pain, diabetic pain or inflammatory pain, pruritus, retinal ischemia/reperfusion damage, macular degeneration, psychiatric disorders, neurodegenerative diseases, cerebral nerve disorders, peripheral nerve disorders, endocrinic disorders such as hyperthyroidism, scarring disorders or wound healing disorders. See, for example U.S. Pat. Nos. 9,328,071; 9,018,383; 8,802,720; 8,618,304; 8,445, 530; and 8,362,073, each of which is incorporated by reference herein in its entirety for disclosures therein of LPA1 receptor antagonists and potential uses thereof.

TRPV1 agonists and antagonists (modulators) have been reported to possibly be therapeutically useful in the treatment or prophylaxis of various disease states, disorders, and conditions associated with TRPV1 activity, including pain, itch, and various inflammatory disorders, inner ear disorders, fever and other disorders or symptoms affected by thermoregulation, tracheobronchial and diaphragmatic dysfunction (including asthma and allergy-related immune responses, cough, bronchospasm, chronic obstructive pulmonary disease, chronic bronchitis, emphysema, and hiccups, gastrointestinal and urinary tract disorders; anxiety eye-related disorders; baldness, and diabetes, among others. See for example, U.S. Pat. Nos. 8,289,397; 8,637,527; 8,673,895; 9,422,293; and 9,440,978, each of which are incorporated by reference herein in its entirety for disclosures therein of TRPV1 antagonists and potential uses thereof.

The present disclosure provides compounds, compositions, the use of compounds and compositions for treating asthma, methods of treatment for asthma, and methods of making medicaments for treating asthma. Treatments relate particularly to those for acute asthma attacks and acute severe asthma attacks to meet the needs in the art.

### SUMMARY

This disclosure generally relates to methods, compounds and pharmaceutical compositions that inhibit or prevent carotid body activation by inflammatory mediators released from the lung during asthma attacks. Inflammatory mediators include among others, cytokines, lipid signalling molecules and/or neuropeptides. Compounds useful in such methods include antagonists to receptors on glomus cells, glia-like sustentacular cells and/or petrosal afferent that are activated by inflammatory mediators and/or reagents that inhibit the carotid body non-specifically by activating carotid body Gi receptors, blocking inward currents or enhancing outward currents.

More particularly the disclosure relates to the use of one or more antagonist of activation of carotid body receptors that are activated/sensitized by lysophosphatidic acid (LPA) and/or by bradykinin released by the lung. More specifically, the disclosure relates to administration of one or more of such antagonists to carotid body receptors that are activated/sensitized by lysophosphatidic acid (LPA) and/or bradykinin released by the lung during an acute asthma attack. Specific antagonists are those of the transient receptor potential cation channel subfamily V member 1 (TRPV1, also known as the capsaicin receptor and/or the vanilloid receptor 1) and a Low density lipoprotein receptor (LPAr, particularly LPAr1-LPAr4). A number of small molecule antagonists of these receptors is known in the art. See, for example, the IUPHAR/BPSA Guide to Pharmacology^{78, 79}. Particular examples of effective TRPV1 antagonists include AMG9810. Particular examples of effective LPAr antagonist include BRP-LPA and Ki16425.

These types of antagonists, singularly or in combination, are useful in the treatment of asthma, and particularly in the treatment of acute asthma (asthma attacks) and severe acute asthma. These types of antagonists are particularly useful in those circumstances where a rescue inhaler or nebulizer do not ameliorate acute or severe acute asthma symptoms. These types of antagonists are also useful for the prevention of an asthma attack, when symptoms or indications would lead one of appropriate medical skill to predict the onset of an attack. Administration may be by any appropriate route. In an embodiment, administration may be by inhalation. In an embodiment, administration may be any route of administration other than inhalation. More specifically, a TRPV1 antagonist, a LPAr antagonist or a combination thereof may be effectively administered by a route other than inhalation, when airway access is severely impeded. In a specific embodiment, administration of the TRVP1 antagonist, LPAr antagonist or a combination thereof is by injection.

In specific embodiments, administration of a combination of a TRPV1 antagonist and an LPAr antagonist prevents asthmatic bronchoconstriction. In specific embodiments, administration of a combination of a TRPV1 antagonist and an LPAr antagonist prevents allergen-induced asthmatic bronchoconstriction. In specific embodiments, prophylactic administration of a combination of a TRPV1 antagonist and an LPAr antagonist prevents asthmatic bronchoconstriction. In specific embodiments, prophylactic administration of a combination of a TRPV1 antagonist and an LPAr antagonist prevents allergen-induced asthmatic bronchoconstriction. In specific embodiments, administration of a combination of a TRPV1 antagonist and an LPAr antagonist administered after allergen exposure ameliorates the associated respiratory distress. In specific embodiments, administration of a combination of a TRPV1 antagonist and an LPAr antagonist administered after allergen exposure can half the associated respiratory distress. In specific embodiments, the TRPV1 antagonist is AMG9810 ((2E)-N-(2,3-Dihydro-1,4-benzodioxin-6-yl)-3-[4-(1,1-dimethylethyl)phenyl]-2-propenamide). In specific embodiments, the LPAr antagonist is BrP-LPA. In specific embodiments, the LPAr antagonist is Ki16425 (3-(4-[4-([1-(2-chlorophenyl)ethoxy]carbonyl amino)-3-methyl-5-isoxazolyl] benzylsulfanyl) propanoic acid or a pharmaceutically acceptable salt thereof. In specific embodiments, the pharmaceutical compositions herein comprises or the methods herein employ a pharmaceutically acceptable salt of a TRPV1 antagonist. In specific embodiments, the pharmaceutical compositions herein comprises or the methods herein employ a pharmaceutically acceptable salt of a LPAr antagonist.

Compounds of the disclosure useful for the methods herein include one or more TRPV1 antagonist, one or more LPAr antagonists or a combinations of one or more TRPV1 antagonists and one or more LPAr antagonists. More specifically, useful compounds include one or more LPAr1 selective antagonists, one or more LPAr2 selective antagonists, one or more LPAr3 selective antagonist, one or more LPAr4 selective antagonists or one or more LPAr6 antagonists. A given LPAr antagonist may be selective for inhibition of a given LPAr or may be an antagonist of two or more or all of LPAr1-LPAr4 and LPAr6. Yet more specifically, useful compounds include one or more LPAr1 selective antagonists, one or more LPAr3 selective antagonist, one or more LPAr4 selective antagonists or one or more LPAr6 antagonists. In specific embodiments, the LPAr antagonist is other than a selective LPAr2 antagonist. The disclosure relates to such antagonist compounds for use in treating asthma, treating acute asthma, treating acute severe asthma, treating refractory asthma, or preventing the onset of acute asthma attacks.

The disclosure provides pharmaceutically acceptable compositions comprising one or more TRPV1 antagonist, one or more LPAr antagonist or a combination of one or more TRPV1 antagonist and one or more LPAr antagonist. In specific embodiments, these pharmaceutically acceptable compositions comprise in addition to the antagonist active ingredient(s), one or more pharmaceutically acceptable excipients or carriers. The disclosure relates to such pharmaceutically acceptable compositions for use in treating asthma, treating acute asthma, treating acute severe asthma, treating refractory asthma, or preventing the onset of acute asthma attacks. In specific embodiments, the pharmaceutically acceptable composition is a composition suitable for inhalation. In specific embodiments, the pharmaceutically acceptable composition is a composition suitable for injection.

The disclosure provides methods for making medicaments for treating asthma, treating acute asthma, treating acute severe asthma, treating refractory asthma, or preventing the onset of acute asthma attacks wherein one or more TRPV1 antagonist, one or more LPAr antagonist or a combination of one or more LPAr antagonist and one or more TRPV1 antagonist are formulated for appropriate administration for treatment of the listed disease or disorder. Medicaments include those for inhalation. Medicaments include those for intravenous administration.

In specific embodiments, one or more TRPV1 antagonist and one or more LPAr antagonist are administered at about the same time to a patient in need thereof. In an embodiment, the one or more TRPV1 antagonist and the one or more LPAr antagonist are formulated separately and administered separately at about the same time. In an embodiment, the one or more TRPV1 antagonist and the one or more LPAr antagonist are formulated together and administered together at the same time. Combined administration includes separate administration of one or more TRPV1 antagonist and one or more LPAr antagonist which are separately formulated, where separate administered is administration of the two types of antagonist by the same or different administrative route, but where administration of the two types of antagonist is timed such that the two types of antagonists exert their biological affect(s) in vivo at about the same time. In a specific embodiment, combined administration includes administration by the same or different administration routes at about the same time. Combined administration also includes administration of a formulation containing one or more TRPV1 antagonist and one or more LPAr antagonist. In specific embodiments, pharmaceutically acceptable compositions are formulated for injection. Pharmaceutically acceptable formulations include solutions or suspensions of the active ingredients in a solvent appropriate for administration to a patient. Solvents include pharmaceutically acceptable aqueous solvents.

In a specific embodiment, combined LPAr and TRPV1 antagonist administration provides acute relief to allergen-induced asthma patients who are refractory to β-agonist therapy, corticosteroids or leukotriene inhibitors. In a specific embodiment, combined LPAr and TRPV1 antagonist treatment provides prophylactic treatment to patients at risk of acute asthma (asthma attacks) and severe acute asthma. The prophylactic affect of this combination treatment can last for, for example, up to 3 or more days.

Other aspects and embodiments of the disclosure will be apparent to one of ordinary skill in the art in view of the drawings, detailed description, and examples.

### BRIEF DESCRIPTION OF THE DRAWINGS

FIGS. 1A-1C: 1A) Effects of TRPV1 and LPAr antagonists on CSN response to LPA. No blockade (1), TRPV1 blockade (AMG9810 10 μM, 2) or LPAr blockade (BrP-LPA 1.5 μM, 3); Ki16425 5 μM, 4). 1B) Summary data. Responses at 2.5 μM LPA: F_{3, 23} (group)=5.031, p=0.01, **. Post-hoc testing-LPA is different from BrP-LPA (p=0.026) and Ki16425 (p=0.018). Responses at 5 μM: F₃, 23 (group)=24.547, p<0.001, ***. Post-hoc testing-LPA is different from BrP-LPA, Ki16425 and AMG9810 (p<0.001); AMG is different from Brp-LPA (p=0.018) and Ki16425 (p=0.044); Responses at 10 μM: F_{3, 23} (group)=14.231, p<0.001, ***. Post-hoc testing-LPA is different from BrP-LPA (p<0.001); Ki16425 (p<0.001); AMG9810 (p=0.022); AMG is different from BrP-LPA (p=0.022). 1C) Summary data of 5 μM LPA (1), with individual TRPV1 blockade (AMG 9810, 10 μM; 2), individual LPAr blockade (BrP-LPA, 1.5 μM; 3); Ki16425, 5 μM; 4), or combined LPAr and TRPV1 blockade (AMG+BrP red; AMG+Ki16425 5), F_{5, 35} (group)=26.164, p<0.001. Post-hoc testing: AMG+LPA is significantly different from Ki16425+AMG+LPA, p=0.005; LPA is significantly different from all other groups, p<0.001, ***.

FIGS. 2A-2B: LPA-mediated carotid sinus nerve excitation. 2A) The application of BrP-LPA (1.5 μM) or 2B) Ki16425 (5 μM) diminishes the response to LPA (5 μM); the remaining response is almost abolished by subsequent application of AMG9810 (10 μM); Dual block portion is denoted in each of FIGS. 2A and 2B.

FIGS. 3A-B: Plasma from ovalbumin-sensitized rats increases carotid body activity in LPA receptor dependent manner. 3A) Carotid sinus nerve activity from a naïve en bloc carotid body preparation in response to plasma from naïve (1) and OVA-sensitized (2) rats. Application of dual LPAr and TRPV1 blockade (AMG9810, 10 μM+BrP-LPA, 1.5 μM) is indicated by the arrow and subsequent trace, 3. 3B) Summary data of the effect of naïve (1) and OVA (2) plasma as well as subsequent dual blockade (3). F2, 17 (group)=40.193, p<0.001, ***. Post hoc testing: OVA vs naïve p<0.001; OVA vs blockade p<0.001.

FIGS. 4A-4B:Bradykinin-induced bronchoconstriction in ovalbumin sensitized rats is dependent on the carotid body and LPA signalling. 4A) Illustration of OVA-sensitization protocol (see Examples, OVA Cohort 2) to test lung-carotid body-lung pathway. 4B) OVA-sensitized and naïve rats were exposed to nebulized saline (baseline) and three consecutive nebulizations of 0.4 mg bradykinin at 1 (solid), 10 (hatched) and 20 (crossed) min while measuring R_{L} and E_{L} (data not shown). Bradykinin had group specific effects: See Examples OVA Cohort 2; F_{14,143} (time×group)=4.035, p<0.001. Post hoc testing; bradykinin caused a marked increase in R_{L} in OVA-sensitized (1; p<0.01, **) but not naïve rats (8; p>0.3) rats; carotid body (CB) denervated (2), vagi (VaG) denervated (3), TRPV1 blockade (AMG9810, 5), LPAr blockade (BrP-LPA, 6), TRPV1 blockade (Ki16425, 7), and dual TRPV1 and LPAr blockade (AMG9810+BrP-LPA, 4), abolished the effects of bradykinin compared to OVA (p<0.01, **; p<0.001, ***).

FIGS. 5A-F. Dual LPAr and TRPV1 blockade abates acute asthmatic respiratory distress in conscious rats. 5A) Illustration of OVA-sensitization protocol to demonstrate respiratory distress in asthmatic model (see Examples OVA Cohort 6). 5B) Inspiratory:expiratory time decreased (Ti:Te; F_{35,431} (time×group)=8.577, p<0.001, post hoc test: p<0.05, *) and 5C) expiratory time increased (Te; F_{35,431} (time×group)=3.948, p<0.001, post hoc test: p<0.05, * difference between groups at indicated time points) in response to acute OVA provocation following OVA sensitization, confirming these parameters as indices of acute asthmatic respiratory distress in conscious animals. 5D) 21-day sensitization and testing protocol to test the effects of dual LPAr and TRPV1 blockade on respiratory distress (see Examples OVA Cohort 7). 5E) Decrease in Ti:Te and 5F) increase in Te caused by allergen provocation are rescued by dual blockade (squares). Ti:Te:F_{70, 1293} (group×time)=3.169, p<0.001; Te: F_{35,385} (time)=10.590, p<0.001, F_{2,35} (group)=7.393, p=0.004). Post-hoc testing-dual block is significantly different from OVA-sensitized saline injected (circles)* and vehicle injected (triangles)^{\hat{ } groups, at indicated time points, p<}0.05. 5G) The peak Ti:Te responses recorded 120 min after OVA exposure on day 21 in animals never having received dual blockade (1, from 5B), having dual blockade on day 21 and recorded on day 21 (Acute treatment 3, from 5E), or having dual blockade on day 18 and recorded on day 21 (Long term treatment, 3, from 5E F_{2,15} (group)=45.805, p<0.001). 5H) The peak Te (120 min) response recorded on day 21 following OVA exposure; groups 1, 2 and 3 as per 5G. Dual antagonist injection on days 18 or 21 reduced respiratory indices of acute bronchoconstriction; and remarkably, dual antagonist injection on day 18 also had beneficial effects three days later, on day 21, without a subsequent dual antagonist injection (F_{2,15} (group)=25.906, p<0.001). Significant difference between indicated groups determined by post hoc testing p<0.001, ***.

### DETAILED DESCRIPTION

This disclosure is based at least in part on the demonstration by the inventors hereof that a systemic increase in lysophosphatidic acid (LPA) released by the lung during asthmatic provocation induces pronounced vagal-mediated bronchoconstriction through stimulation of the carotid bodies (main peripheral autonomic chemoreceptors). More specifically, carotid body activation during airborne allergic provocation is demonstrated to be caused by systemic release of LPA from the lung. This carotid body activation is sufficient to cause acute bronchoconstriction. This disclosure confirms the systemic upregulation of lysophosphatidic acid in response to allergen provocation and the importance of carotid bodies to asthmatic attacks. It is demonstrated that LPA activates the carotid body, via transient receptor potential 1 (TRPV1) and LPA-specific receptors, which in turn activates vagal efferents. Moreover, the carotid body-mediated pathway is found to form a significant component of acute asthmatic bronchoconstriction. This mechanism has medical importance. Blocking both TRPV1 and LPA-specific receptors suppresses asthmatic airway bronchoconstriction in response to immune challenge with ovalbumin. This disclosure provides new evidence linking inflammatory mediators to a novel reflex pathway inducing bronchoconstriction. In an embodiment, the disclosure provides at least a new form of emergency therapy to mitigate asthmatic attacks.

The inventors have also demonstrated that lysophosphatidic acid stimulates the carotid body (CB): linking inflammation and carotid sinus nerve activity. Systemic levels of the inflammatory mediator lysophosphatidic acid (LPA) increase with inflammatory lung disease⁷³, LPA is a TRPV1 agonist⁷¹ and TRPV1 is expressed in CB⁴¹. The inventors evaluated the effects of LPA on carotid sinus nerve and phrenic nerve discharge in novel en bloc carotid body perfused and in situ decerebrate, vagotomized, rodent dual-perfused preparations, respectively (see Examples). In carotid sinus nerve intact and denervated in situ preparations, carotid bodies and the brainstem were perfused separately. The CB were perfused with normoxia/normocapnia and the brainstem was perfused with hypocapnia to induce apnea and thus increase phrenic burst frequency sensitivity to carotid body activation. LPA (5 μM) was injected into the carotid body perfusate. To determine whether non-TRPV1 LPA receptors might also be involved, the effects of LPA were tested in the presence of the TRPV1 antagonist AMG9810 using the en bloc preparation.

LPA delivered directly to the CB circulation in in situ preparations significantly (p<0.05) increased phrenic nerve burst rate (from 1.2±0.8 burst·min⁻¹ to 20.8±2.9 burst·min⁻¹) and amplitude (from 1±0.5 to 2.5±0.5 normalized units). In CB denervated preparations, LPA had no effect. Blocking TRPV1 receptors reduced but did not eliminate the effects of LPA on carotid sinus nerve activity in the en bloc preparation.

RT-PCR revealed expression of LPAr 1, 3, and 4 in the carotid body, LPAr 3 in the petrosal ganglia and LPArr 1, and 3 in the superior cervical ganglia. The data indicated that the CB are sensitive to LPA via TRPV1 and LPAr receptors and involved in the neural components of inflammatory pulmonary diseases.

TRPV1 receptor is a member of a family of TRP receptors and is broadly expressed in various tissues which contact the environment (e.g., skin, gut, airways). TRPV1 is reported to be activated by various agents including capsaicin among which chemical irritants, inflammatory mediators and tissue damaging stimuli can be identified. TRPV1 is also reported to be activated by high temperature (>43° C.), acidic pH (<5.3), intracellular redox states and electrostatic charges⁸⁰. Modulators (particularly antagonists) of TRPV1, and particularly those modulators and antagonists that are selective for TRPV1 over other TRP receptor family members, have, for example, been investigated as therapeutic targets for treatment of pain and inflammation. TRPV1 antagonists are reported to be effective in models of inflammatory, osteoarthritis and neuropathic pain. Kort and Kym provide a review of TRPV1 antagonists as clinical targets⁸¹. This reference provides description of a number of TRPV1 antagonists with in some cases descriptions of the results of clinical trials. The authors note that the clinical application of first generation TRPV1 clinical leads has been made problematic by the undesired side effect of increased core body temperature associated with administration of TRPV1 antagonists. The authors, however, also report that more recent identification of TRPV1 antagonists that do not exhibit the undesired core body increase and as such are called temperature-neutral. Examples of such temperature neutral TRPV1 antagonists are provided. The authors note TRPV1 antagonists which differentially block TPRV1 activation by different stimuli, e.g., block capsaicin activation but not heat or proton activation. TRPV1 antagonists which exhibit differentiated blockage (stronger) of capsaicin-induced calcium efflux, compared to blockage (weaker) of acid-induced TRPV1 are associated with temperature-neutrality.

For certain clinical applications as described herein which involve short-term administration (hours, or days) of a TRPV1 antagonist to treat symptoms of asthma attacks, it is presently believed that undesired effects on core body temperature are not a significant detriment to use of the antagonist. However, in certain embodiments herein, the TRPV1 antagonists employed for treatment of asthma, acute asthma and severe acute asthma are temperature-neutral TRPV1 antagonists as described in Kort and Kym⁸¹ and references cited therein, which exhibit little or no effect on core body temperature and are thus temperature-neutral. This reference is incorporated by reference herein in its entirety for names and structures of useful TRPV1 antagonists for applications as described herein. An exemplary temperature neutral TRPV1 antagonist is AS1928379⁸². Another exemplary temperature neutral TRPV1 antagonist is A1165442⁸³.

Wong and Gavva⁸⁴ is another review of therapeutic potential of TRPV1 agonists and antagonists which appears to focus on analgesic applications. This reference is incorporated by reference herein in its entirety for descriptions of TRPV1 antagonists which may be useful in applications herein.

LPA is a phospholipid signalling molecule that is reported to activate at least five known G protein-coupled receptors (GPCRs), designated LPA1-LPA5⁸⁵. A sixth receptor designated LPA6 has been described⁸⁶. LPA is associated with a variety of developmental, physiological, and pathophysiological effects. LPAr have been identified as targets for the treatment of important diseases including neuropsychiatric disorders, neuropathic pain, infertility, cardiovascular disease, inflammation, fibrosis, and cancer. LPA1 shares significant amino acid sequences identity with LPA2 and LPA3. LPA4, LPA5 and LPA6 are more diverse in sequence to the other receptors in the family and to each other. Most known LPA antagonists inhibit LPA1, LPA2 and LPA3. LPA is reported to be a potent mediator of immune response. A number of LPA antagonists are reported in Yung et al.⁸⁶ where in Table 2, therein, a summary of LPA receptor modulators, receptor subtype target and activity and disease relevance are listed. This reference is incorporated by reference herein in its entirety for descriptions of LPAr antagonists and their selectivity and activity.

This disclosure relates to compounds, compositions and methods for treating asthma, particularly acute asthma and severe acute asthma and the prevention of acute and acute severe asthma attacks. The methods employ one or more TRPV1 antagonist or one or more LPAr antagonist and preferably employ a combination of one or more TRPV1 antagonist and one or more LPAr antagonists. Preferred LPAr antagonists are those that are antagonists of LPAr1, LPAr2, LPAr3, LPAr4 and/or LPAr6. Further preferred LPAr antagonists are those that are antagonists of LPAr1, LPAr2, LPAr3 and/or LPAr6. Further preferred LPAr antagonists are those that are antagonists of at least LPAr1. Further preferred LPAr antagonists are those that are antagonists of at least LPAr3. Further preferred LPAr antagonists are those that are antagonists of at least LPAr4. Further preferred LPAr antagonists are those that are antagonists of at least LPAr6. Further preferred LPAr antagonists are those that are antagonists of at least LPAr2. Further preferred LPAr antagonists are those that are antagonists of at least LPAr1, LPAr3, and LPAr4. Further preferred LPAr antagonists are those that are antagonists of LPAr other than selective antagonists of LPAr2. Selective LPAr antagonists are useful in the methods herein. In specific embodiments, preferred selective LPAr antagonists are those that are selective antagonists of LPAr1. In specific embodiments, preferred selective LPAr antagonists are those that are selective antagonists of LPAr2. In specific embodiments, preferred selective LPAr antagonists are those that are selective antagonists of LPAr3. In specific embodiments, preferred selective LPAr antagonists are those that are selective antagonists of LPAr4. In specific embodiments, preferred selective LPAr antagonists are those that are selective antagonists of LPAr6. In specific embodiments, selective LPAr antagonists are those that are selective antagonists of LPAr1, LPAr3 and LPAr4. In specific embodiments, selective LPAr antagonists are those that are selective antagonists of LPAr1, LPAr2, LPAr3 and LPAr4. In specific embodiments, selective LPAr antagonists are those that are selective antagonists of LPAr5 and/or LPAr6.

TRPV1 antagonists are known in the art and are available from commercial sources or can be prepared from known readily available starting materials and reagents using known methods or routine adaptations of known methods. LPAr antagonists are known in the art and are available from commercial sources or can be prepared from known readily available starting materials and reagents using known methods or routine adaptations of known methods. This disclosure provides the names of a variety of useful antagonists. The chemical structures of named antagonists are known in the art and in many cases provided in references cited herein. Methods are also known in the art for identifying new TRPV1 antagonists and new LPAr antagonists. It will be appreciated that such newly identified antagonists can be employed in the methods described herein.

The terms “TRPV1 antagonist” and “LPAr antagonist” are used generally as these terms are used in the art. Preferred for use in methods and compositions herein are those antagonists that are pharmaceutically acceptable. Antagonists include pharmaceutically acceptable salts of TRPV1 antagonists and LPAr antagonists. Also preferred for use in methods and compositions herein are antagonists that are small molecules of molecular weight less than 900 daltons and more specifically of 500 daltons or less. Small molecule antagonists are generally organic compounds which may be isolated from natural sources or which are synthetic non-naturally-occurring organic molecules.

Exemplary useful TRPV1 antagonists are provided in Tables 1 and 2. Exemplary useful LPAr antagonists are provided in Tables 3 and 4. Any one or more of the antagonists identified in the Tables 1-4 can be used in the compositions and methods herein.

Selective LPAr antagonists are known in the art. For example, compound 15 of the reference Beck et al.⁸⁷ is reported to be an LAPR2 selective antagonist. Compound 12 of reference Fells et al. ⁸⁸ is reported to be an LAPR3 selective antagonist. Selective LPAr antagonists can be used in the compositions and methods herein.

Additional exemplary TRPV1 antagonists are reported in U.S. Pat. Nos. 9,440,978; 9,422,293; 9,029,378; 8,901,155; 8,815,930; 8,748,610; 8,765,815; 8,691,855; 8,637,527; 8,557,872; 8,383,839; 8,350,083; 8,343,971; 8,338,603; 8,232,309; 8,211,927; 8,030,504; 7,960,584; 7,919,624; 7,910,751; 7,858,621; 7,767,705; 7,632,519; and 7,482,469. Each of these patents is incorporated by reference herein in its entirety for descriptions of TRPV1 antagonists and methods of preparing such antagonists.

Additional exemplary LPAr antagonists are reported in U.S. Pat. Nos. 9,624,182; 9,556,133; 9,527,850; 9,346,762; 9,272,940; 9,090,573; 9,067,938; 9,018,383; 8,859,775; 8,785,442; 8,778,983; 8,686,177; 8,664,220; 8,592,402; 8,541,587; 8,455,499; 8,440,707; 8,362,073; 8,283,339; 8,124,645; 7,947,665; 7,820,703; and 7,217,704. Each of these patents in incorporated by reference herein in its entirety for descriptions of LPAr antagonists and methods of preparing such antagonists. One or more of these patents has description with respect to selective LPAr antagonists.

TABLE 1
Exemplary Trpv1 Antagonists^{#}

2-APB (2-Aminoethoxydiphenyl borate)
5′-IRTX
6-iodo-nordihydrocapsaicin
AA-5-HT
A1165442
A425619
A778317
AMG517
AMG628
AMG8562
AMG9810
AMG21629
AS1928370
BCTC
Capsazepine
JNJ17203212
JYL1421
L-R4W2
NADA
α-spinasterol
SB366791
SB452533
SB705498
^{#}Chemical names/structures of most of the TPRV1 antagonist in this list can be found in reference 78. The structures of several of the compounds are found in specific references included in Table 2.

TABLE 2
Preferred TRPV1 antagonists:

A 784168: N-5-Isoquinolinyl-N′-[[(4-(trifluoromethyl)phenyl]methyl]urea (A 425619)
3,6-Dihydro-3′-(trifluoromethyl)-N-[4-[(trifluoromethyl)sulfonyl]phenyl]-[1(2H),2′-
bipyridine]-4-carboxamide:
Bianchi et al (2007) [3H]-A-778317 [1-((R-5-tert-butyl-indan-1-yl)-3-
isoquinolin-5-yl-urea]: a novel, stereoselective, high-affinity antagonist is a
useful radioligand for the human transient receptor potential vanilloid-1
(TRPV1) receptor. J. Pharmacol. Exp. Ther. 323 285. PMID: 17660385.
Cui et al (2006) TRPV1 receptors in the CNS play a key role in broad-
spectrum analgesia of TRPV1 antagonists. J. Neurosci. 26 9385. PMID:
16971522.
AMG21629: 3-Amino-5-[[2-[(2-methoxyethyl)amino]-6-[4-(trifluoromethyl)phenyl]-4-
pyrimidinyl]oxy]-2(1H)-quinoxalinone:
Tamayo et al (2008) Design and synthesis of peripherally restricted transient
receptor potential vanilloid 1 (TRPV1) antagonists. J. Med. Chem. 51 2744.
PMID: 18386885.
Gavva et al (2007) The vanilloid receptor TRPV1 is tonically activated in vivo
and involved in body temperature regulation. J. Neurosci. 27 3366. PMID:
17392452.
AMG517: N-[4-[[6-[4-(Trifluoromethyl)phenyl]-4-pyrimidinyl]oxy]-2-
benzothiazolyl]acetamide:
Doherty et al (2007) Novel vanilloid receptor-1 antagonists: 2. Structure-
activity relationships of 4-oxopyrimidines leading to the selection of a clinical
candidate. J. Med. Chem. 50 3515. PMID: 17585750.
Gavva et al (2007) Repeated administration of vanilloid receptor TRPV1
antagonists attenuates hyperthermia elicited by TRPV1 blockade.
J. PharmacoI. Exp. Ther. 323 128. PMID: 17652633.
Wang et al (2007) Novel vanilloid receptor-1 antagonists: 3. The identification
of a second-generation clinical candidate with improved physicochemical and
pharmacokinetic properties. J. Med. Chem. 50 3528. PMID: 17585751.
AMG8562: (R,E)-N-(2-hydroxy-2,3-dihydro-1H-inden-4-yl)-3-(2-(piperidin-1-yl)-4-
(trifluoromethyl)phenyl)-acrylamide
Lehto SG et al. (2008) J Pharmacol Exp Ther 326: 218-229
AMG9810: (2E)-N-(2,3-Dihydro-1,4-benzodioxin-6-yl)-3-[4-(1,1-
dimethylethyl)phenyl]-2-propenamide:
Doherty et al (2005) Discovery of potent, orally available vanilloid receptor-1
antagonists. Structure-activity relationship of N-aryl cinnamides.
J. Med. Chem. 48 71. PMID: 15634002.
Gavva et al (2005) AMG 9810 [(E)-3-(4-t-Butylphenyl)-N-(2,3-
dihydrobenzo[b][1,4]dioxin-6-yl)acrylamide], a novel vanilloid receptor 1
(TRPV1) antagonist with antihyperalgesic properties. J. Pharmacol. Exp. Ther.
313 474. PMID: 15615864.
AA-5-HT: N-[2-(5-Hydroxy-1H-indol-3-yl)ethyl]-5,8,11,14-eicosatetraenamide
(Alternative name: Arachidonyl serotonin):
Maione et al (2007) Analgesic actions of N-arachidonoyl-serotonin, a fatty
acid amide hydrolase inhibitor with antagonistic activity at vanilloid TRPV1
receptors. Br. J. Pharmacol. 150 766. PMID: 17279090.
Di Marzo et al (2004) The endocannabinoid system and its therapeutic
exploitation. Nat. Rev. Drug Discov. 3 771. PMID: 15340387.
Bisogno et al (1998) Arachidonoylserotonin and other novel inhibitors of fatty
acid amide hydrolase. Biochem. Biophys. Res. Comms. 248 515.
AS1928370: (R)-N-(1-methyl-2-oxo-1,2,3,4-tetrahydro-7-quinolyl)-2-[(2-
methylpyrrolidin-1-yl)methyl]biphenyl-4-carboxamide
Watabiki T, Kiso T, Tsukamoto M, Aoki T, Matsuoka N. Biol Pharm Bull.
2011; 34(7): 1105-8.
Watabiki T, Kiso T, Kuramochi T, Yonezawa K, Tsuji N, Kohara A, Kakimoto
S, Aoki T, Matsuoka N. J Pharmacol Exp Ther. 2011 March; 336(3): 743-50. doi:
10.1124/jpet.110.175570.
A1165442: (R)-1-(7-chloro-2,2-bis(fluoromethyl)chroman-4-yl)-3-(3-
methylisoquinolin-5-yl)urea (A-1165442)
Regina M. Reilly et al. (2012) J. Pharmacology and Experimental
Therapeutics. 342 (2) 416-428.
BCTC: 4-(3-Chloro-2-pyridinyl)-N-[4-(1,1-dimethylethyl)phenyl]-1-
piperazinecarboxamide:
Valenzano, K. J., Grant, E. R., Wu, G., et al. N-(4-tertiarybutylphenyl)-4-(3-
chloropyridin-2-yl)tetrahydropyrazine-1 (2H)-carbox-amide (BCTC), a novel
orally effective vanilloid receptor 1 antagonist with analgesic properties: I. In
vitro characterization and pharmacokinetic properties. Journal of
Pharmacology and Experimental Therapeutics 306(1), 377-386 (2003).
Gavva, N. R., Tamir, R., Klionsky, L., et al. Proton activation does not alter
antagonist interaction with the capsaicin-binding pocket of TRPV1. Molecular
Pharmacology 68(6), 1524-1533 (2005).
5′-IRTX: 6,7-Deepoxy-6,7-didehydro-5-deoxy-21-dephenyl-21-(phenylmethyl)-
daphnetoxin,20-(4-hydroxy-5-iodo-3-methoxybenzeneacetate) (Alternative
Names: 5′-Iodoresiniferatoxin, Iodoresiniferatoxin):
Marinelli et al (2002) Capsaicin activation of glutamatergic synaptic
transmission in the rat locus coeruleus in vitro. J. Physiol. 543 531. PMID:
12205187.
Seabrook et al (2001) Properties of iodo-resiniferatoxin - a high affinity VR1
vanilloid receptor antagonist. Soc. Neurosci. Abstr. 925.9.
Wahl et al (2001) Iodo-resiniferatoxin, a new potent vanilloid receptor
antagonist. Mol. Pharmacol. 59 9. PMID: 11125018.
JNJ 172032124: [3-(Trifluoromethyl)-2-pyridinyl]-N-[5-(trifluoromethyl)-2-pyridinyl]-1-
piperazinecarboxamide:
Bhattacharya et al (2007) Pharmacology and antitussive efficacy of 4-(3-
trifluoromethyl-pyridin-2-yl)-piperazine-1-carboxylic acid (5-trifluoromethyl-
pyridin-2-yl)-amide (JNJ17203212), a transient receptor potential vanilloid 1
antagonist in guinea pigs. J. Pharmacol. Exp. Ther. 323 665. PMID: 17690251.
Ghilardi et al (2005) Selective blockade of the capsaicin receptor TRPV1
attenuates bone cancer pain. J. Neurosci. 25 3126. PMID: 15788769.
Swanson et al (2005) Identification and biological evaluation of 4-(3-
trifluoromethylpyridin-2-yl)piperazine-1-carboxylic acid (5-
trifluoromethylpyridin-2-yl)amide, a high affinity TRPV1 (VR1) vanilloid
receptor antagonist. J. Med. Chem. 48 1857. PMID: 15771431.
L-R₄W₂ (Arginine-rich hexapeptide):
Himmel et al (2002) The arginine-rich hexapeptide R4W2 is a stereoselective
antagonist at the vanilloid receptor 1: a Ca2+ imaging study in adult rat dorsal
root ganglion neurons. J. Pharmacol. Exp. Ther. 301 981. PMID: 12023528.
Planells-Cases et al (2000) Arginine-rich peptides are blockers of VR-1
channels with analgesic activity. FEBS Lett. 481 131. PMID: 10996311.
SB366791: 4′-Chloro-3-methoxycinnamanilide:
Gavva et al (2005) Proton activation does not alter antagonist interaction with
the capsaicin-binding pocket of TRPV1. Mol. Pharmacol. 68 1524. PMID:
16135784.
Gunthorpe et al (2004) Identification and characterisation of SB-366791, a
potent and selective vanilloid receptor (VR1/TRPV1) antagonist.
Neuropharmacology 46 133. PMID: 14654105.
Fowler et al (2003) Inhibition of C6 glioma cell proliferation by anandamide,
1-arachidonylglycerol, and by a water soluble phosphate ester of
anandamide: variability in response and involvement of arachidonic acid.
Biochem. Pharmacol. 66 757. PMID: 12948856.
SB452533: N-(2-Bromophenyl)-N′-[2-[ethyl(3-methylphenyl)amino]ethyl]-urea:
Bianchi et al (2007) [3H]A-778317 [1-((R)-5-tert-Butyl-indan-1-yl)-3-
isoquinolin-5-ylurea]: a novel, stereoselective, high-affinity antagonist is a
useful radioligand for the human transient receptor potential vanilloid-1
(TRPV1) receptor. J. Pharmacol. Exp. Ther. 323 285. PMID: 17660385.
Weil et al (2005) Conservation of functional and pharmacological properties
in the distantly related temperature sensors TRPV1 and TRPM8.
Mol. Pharmacol. 68 518. PMID: 15911692.
Rami et al (2004) Discovery of small molecule antagonists of TRPV1.
Bioorg. Med. Chem. Lett. 14 3631. PMID: 15203132.
α-Spinasterol:(3β,5α,22E)-Stigmasta-7.22-dien-3-ol
Trevisan et al (2012) Identification of the plant steroid α-spinasterol as a
novel transient receptor potential vanilloid 1 antagonist with antinociceptive
properties. J. Pharmacol. Exp. Ther. 343 258. PMID: 22837009.

TABLE 3
Exemplary LPAr Antagonists#

AM095
Am966
BMS986020
Ki16425-3-(4-[4-([1-(2-chlorophenyl)ethoxy]carbonyl
amino)-3-methyl-5-isoxazolyl] benzylsulfanyl)
propanoic acid
ONO-3080573
ONO-7300243
ONO-9780307
ONO-9910539
VPC 12249
VPC 32179
VPV 32183
BrP-LPA-[(3S)-1-bromo-4-hexadecanoyloxy-3-
hydroxybutyl]phosphonic acid
Syn BrP-LPA
Anti BrR-LPA
Dioctanoylglycerolpyrophosphate
Farnesyl monophosphate
Farnesyl diphosphate
Dodecyl thiophosphate
1-bromo-(3S)-hydroxy-4-(palmitoyl) butyl phosphate
#Chemical names/structures of most of the LPAr antagonist in this table can be found in reference 89. Reference 89 also provides lists of possibly selective LPAr antagonists.

TABLE 4
Additional LPAr Antagonists

H2L 5765834: 2,3-Dihydro-2-[3-(4-nitrophenoxy)phenyl]-1,3-dioxo-1H-isoindole-5-carboxylic
acid:
Fells et al (2010) 2D binary QSAR modeling of LPA3 receptor antagonism. J. Mol. Graph
Model. 28 828. PMID: 20356772.
Tigyi (2010) Aiming drug discovery at lysophosphatidic acid targets. Br. J. Pharmacol.
161 241. PMID: 20735414.
Williams et al (2009) Unique ligand selectivity of the GPR92/LPA5 lysophosphatidate
receptor indicates role in human platelet activation. J. Biol. Chem. 284 17304. PMID:
19366702.
H2L5186303: (Z,Z)-4,4′-[1,3-Phenylenebis(oxy-4,1-phenyleneimino)]bis[4-oxo-2-butenoic acid:
Fells et al (2009) Structure-based drug design identifies novel LPA3 antagonists.
Bioorg. Med. Chem. 17 7457. PMID: 19800804.
Fells et al (2008) Identification of non-lipid LPA3 antagonists by virtual screening.
Bioorg. Med. Chem. 16 6207. PMID: 18467108.
Ro 6842262: 1-[4′-[4-Methyl-5-[[[(1R)-1-phenylethoxy]carbonylamino]-1H-1,2,3-triazol-1-yl][1,1′-
biphenyl]-4-yl]cyclopropanecarboxylic acid:
Qian et al (2012) Discovery of highly selective and orally active lysophosphatidic acid
receptor-1 antagonists with potent activity on human lung fibroblasts. J. Med. Chem. 55
7920. PMID: 22894757.
Spiroxatrine: 8-[(2,3-Dihydro-1,4-benzodioxin-2-yl)methyl]-1-phenyl-1,3,8-
triazaspiro[4,5]decan-4-one:
Bylund et al (1992) Pharmacological characteristics of α2-adrenergic receptors:
comparison of pharmacologically defined subtypes with subtypes identified by molecular
cloning. Mol. Pharmacol. 42 1. PMID: 1353247.
Schoeffter and Hoyer (1988) Centrally acting hypotensive agents with affinity for 5-HT1A
binding sites inhibit forskolin-stimulated adenylate cyclase activity in calf hippocampus.
Br. J. Pharmacol. 95 975. PMID: 3207999.
Nelson and Taylor (1986) Spiroxatrine: a selective serotonin 1A receptor antagonist.
Eur. J. Pharmacol. 124 207. PMID: 3720840.
TC LPA5 4: 5-(3-Chloro-4-cyclohexylphenyl)-1-(3-methoxyphenyl)-1H-pyrazole-3-carboxylic
acid
Kozian et al (2012) Selective non-lipid modulator of LPA5 activity in human platelets.
Biorg. Med. Chem. Lett. 22 5239. PMID: 22801643.

In specific embodiments, the disclosure provides methods for treatment of acute asthmatic attack or for prevention of an acute attack that is predicted.

In specific embodiments, treatment involves administration of one or more antagonist targeting the family of carotid body receptors activated by LPA (e.g. AMG9810 and/or BrP-LPA), and optionally one or more antagonist targeting carotid body receptors activated by LPA and/or other substances released by the lung during an asthmatic attack (e.g. bradykinin) and/or optionally one or more reagent that inhibits LPA stimulation of carotid body through LPA-independent mechanisms (e.g., somatostatin or other natural substances that inhibit the carotid body; blockers of endogenous channels or receptors causing inward currents; activators of endogenous channels or receptors causing outward current; activation of artificial channels or receptors causing outward currents).

In specific embodiments, the route of administration may be by inhalation, injection (syringe, autoinjector), adsorption through skin/mucus membrane, suppository, patch or ingestion. In specific embodiments, the route of administration is other than by inhalation.

Compounds of the disclosure may contain chemical groups (acidic or basic groups) that can be in the form of salts. Pharmaceutically acceptable salts of antagonists of this disclosure can be employed in the compositions and methods herein. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts, alkaline earth metal salts such as calcium and magnesium salts, salts with organic bases (e.g., organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, salts with amino acids such as arginine, lysine and the like and salts with amino sugars, such as meglumine. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Salt of the disclosure include “pharmaceutically acceptable salts” which refers to those salts which retain the biological effectiveness and properties of the free bases or free acids, and which are not biologically or otherwise undesirable and acceptable for use in pharmaceutical compositions. Pharmaceutically acceptable salts comprise pharmaceutically-acceptable anions and/or cations.

Compounds of the present disclosure, and salts thereof, may exist in their tautomeric form, in which hydrogen atoms are transposed to other parts of the molecules and the chemical bonds between the atoms of the molecules are consequently rearranged. It should be understood that all tautomeric forms, insofar as they may exist, are included within the disclosure. Additionally, disclosed compounds may have trans and cis isomers and may contain one or more chiral centers, therefore exist in enantiomeric and diastereomeric forms. The disclosure includes all such isomers, as well as mixtures of cis and trans isomers, mixtures of diastereomers and racemic mixtures of enantiomers (optical isomers). When no specific mention is made of the configuration (cis, trans or R or S) of a compound (or of an asymmetric carbon), then any one of the isomers or a mixture of more than one isomer is intended. The processes for preparation can use racemates, enantiomers, or diastereomers as starting materials. When enantiomeric or diastereomeric products are prepared, they can be separated by conventional methods, for example, by chromatographic or fractional crystallization. The inventive compounds may be in the free or hydrate form. The term enantiomerically pure refers to a sample containing molecules of a given structure whose molecules have the same chirality sense (i.e., are the same optical isomer) within the limits of detection. The term substantially enantiomerically pure refers to a sample containing molecules of a given structure, wherein equal to or less than 1% of the molecules of the sample have a different chirality sense. Compounds of the invention include those which are enatiomerically pure and those that are substantially enatiomerically pure.

The disclosure provides pharmaceutical compositions for use in the treatment methods herein. Pharmaceutical compositions comprise one or more of the active antagonists as described optionally in combination with a pharmaceutically acceptable carrier. Pharmaceutically acceptable carriers are those carriers that are compatible with the other ingredients in the formulation and are biologically acceptable. Carriers can be solid or liquid. Solid carriers can include one or more substances that can also act as flavoring agents, lubricants, solubilizers, suspending agents, fillers, glidants, compression aids, binders, tablet-disintegrating agents, or encapsulating materials. Liquid carriers can be used in preparing solutions, suspensions, emulsions, syrups and elixirs. The active ingredient can be dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Compositions for oral administration can be in either liquid or solid form.

Suitable solid carriers include, for example, alumina, calcium phosphate, ion exchange material, aluminum or magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Suitable examples of liquid carriers for oral and parenteral administration include water of appropriate purity, aqueous solutions (particularly containing additives, e.g. cellulose derivatives, sodium carboxymethyl cellulose solution), alcohols (including monohydric alcohols and polyhydric alcohols e.g. glycols) and their derivatives, and oils. For parenteral administration, the carrier can also be an oily ester such as ethyl oleate and isopropyl myristate. Sterile liquid carriers are used in sterile liquid form compositions for parenteral administration. Compositions for administration can be solutions or suspensions. The liquid carrier for pressurized compositions can be halogenated hydrocarbon or other pharmaceutically acceptable propellant. Liquid pharmaceutical compositions that are sterile solutions or suspensions can be administered parenterally by, for example, intramuscular, intraperitoneal or subcutaneous injection. Sterile solutions can also be administered intravenously. Compositions for oral administration can be in either liquid or solid form.

Liquid or solid pharmaceutical compositions can contain one or more of buffering agents (e.g., phosphates and/or hydrogen phosphate salts), salts (e.g., NaCl, zinc salts), electrolytes, osmotic agents (e.g., glycerol), protamine sulfate, polymeric materials (such as pyrrolidone, cellulose and cellulose derivatives, polyethylene glycol, polyacrylates, and polyethylene glycol). Liquid solutions, suspensions, gels, creams and the like may include surface active ingredients (including among others dispersing agents, wetting agents, suspending agents, etc.)

Solid pharmaceutical compositions may contain among others, colloidal silica, magnesium trisilicate, glycerides, fatty acids and salts thereof. Carriers for oral dosage forms (e.g., tablets and capsules) include among others lactose and corn starch.

Methods of this disclosure comprise the step of administering a “therapeutically effective amount” of the present therapeutic formulations containing one or more of the present active compounds, to treat, reduce or regulate a disease state in a patient. A given therapeutic formulation may also prevent the onset of a disease or disorder, slow the development of the disease or disorder or ameliorate one or more symptoms of the disease or disorder. As is understood in the art, the therapeutically effective amount of a given compound or formulation will depend at least in part upon, the mode of administration (e.g., intravenous, oral, topical administration), any carrier or vehicle employed, and the specific individual to whom the formulation is to be administered (age, weight, condition, sex, etc.). The dosage requirements need to achieve the “therapeutically effective amount” vary with the particular formulations employed, the route of administration, and clinical objectives. Based on the results obtained in standard pharmacological test procedures, projected daily dosages of active compound can be determined as is understood in the art.

The methods of treatment and prophylaxis herein are useful in treating animals, particularly mammals, and more particularly in treating humans. The methods, compositions, medicaments and kits herein can be applied for human or veterinary applications. Human applications are generally to any human susceptible to or suffering from asthma and particularly those humans at risk of acute asthma attacks. Veterinary applications are generally to any animal susceptible to or suffering from asthma. In particular, the methods, compositions, medicaments and kits herein can be used to treat bovine or equine asthma.

Exemplary daily dosage levels of individual active ingredients or combinations thereof in a combined formulation range from 0.001 to 100 mg/kg body weight or more specifically from 1 to 10 mg/kg body weight.

Any suitable form of administration can be employed in the methods herein. Administration includes any form of administration that is known in the art and is intended to encompass administration in any appropriate dosage form and further is intended to encompass administration of a compound, alone or in a pharmaceutically acceptable carrier. Pharmaceutical carriers are selected as is known in the art based on the chosen route of administration and standard pharmaceutical practice. The compounds of this disclosure can, for example, be administered in oral dosage forms including tablets, capsules, pills, powders, granules, elixirs, tinctures, suspensions, syrups and emulsions. Oral dosage forms may include sustained release or timed release formulations. The compounds of this disclosure may also be administered topically, intravenously, intraperitoneally, subcutaneously, or intramuscularly, all using dosage forms well known to those of ordinary skill in the pharmaceutical arts. Compounds of this disclosure can also be administered in intranasal form by topical use of suitable intranasal vehicles. For intranasal or intrabronchial inhalation or insulation, the compounds of this disclosure may be formulated into an aqueous or partially aqueous solution, which can then be utilized in the form of an aerosol. In specific embodiments, a form of administration that is other than inhalation is employed.

The compounds of this disclosure may be administered rectally or vaginally in the form of a conventional suppository. The compounds of this disclosure may also be administered transdermally through the use of a transdermal patch containing the active compound and a carrier that is inert to the active compound, is non-toxic to the skin, and allows delivery of the agent for systemic absorption into the blood stream via the skin.

Pharmaceutical compositions and medicaments of this disclosure comprise one or more compounds of the disclosure of formula I (or other formulas herein) in optional combination with a pharmaceutically acceptable carrier, excipient, or diluent. Such compositions and medicaments are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. The disclosure also encompasses method for making a medicament employing one or more compounds of this disclosure which exhibit a therapeutic effect.

Compounds useful in the methods of this disclosure include pharmaceutically-acceptable salts of the compounds of formulas herein. Compounds useful in the methods of this disclosure include pharmaceutically-acceptable prodrugs of the compounds of formulas herein. Salts include any salts derived from the acids of the formulas herein which are acceptable for use in human or veterinary applications.

In a preferred embodiment, pharmaceutical compositions herein comprise a combination of a TRPV1 antagonist and an LPAr antagonist. In specific embodiments, the pharmaceutical composition comprises a TRPV1 antagonist of Table 2 and an LPAr antagonist of Table 4. In specific embodiments, the molar ratio of TRPV1 antagonist to LPAr antagonist in the composition ranges from 50:1 to 1:50. In specific embodiments, the molar ratio of TRPV1 antagonist to LPAr antagonist in the composition ranges from 20:1 to 1:20. In specific embodiments, the molar ratio of TRPV1 antagonist to LPAr antagonist in the composition ranges from 10:1 to 1:10. In specific embodiments, the molar ratio of TRPV1 antagonist to LPAr antagonist in the composition ranges from 10:1 to 1:1. In specific embodiments, the molar ratio of TRPV1 antagonist to LPAr antagonist in the composition ranges from 10:1 to 2:1. In specific embodiments, the molar ratio of TRPV1 antagonist to LPAr antagonist in the composition ranges from 5:1 to 2:1.

In specific embodiments, the disclosure provides methods of making medicaments wherein the medicaments are pharmaceutical compositions comprising a combination of a TRPV1 antagonist and an LPAr antagonist. In specific embodiments, the medicament comprises a TRPV1 antagonist of Table 2 and an LPAr antagonist of Table 4. In specific embodiments, the molar ratio of TRPV1 antagonist to LPAr antagonist in the medicament ranges from 50:1 to 1:50. In specific embodiments, the molar ratio of TRPV1 antagonist to LPAr antagonist in the medicament ranges from 20:1 to 1:20. In specific embodiments, the molar ratio of TRPV1 antagonist to LPAr antagonist in the medicament ranges from 10:1 to 1:10. In specific embodiments, the molar ratio of TRPV1 antagonist to LPAr antagonist in the medicament ranges from 10:1 to 1:1. In specific embodiments, the molar ratio of TRPV1 antagonist to LPAr antagonist in the medicament ranges from 10:1 to 2:1. In specific embodiments, the molar ratio of TRPV1 antagonist to LPAr antagonist in the medicament ranges from 5:1 to 2:1. In an embodiment, the medicament comprises a TRPV1 antagonist separately formulated from a LPAr antagonist. In an embodiment, the medicament comprises a TRPV1 antagonist formulated together with a LPAr antagonist. In an embodiment, the medicament comprises a TRPV1 antagonist formulated together with a LPAr antagonist for administration by injection. In an embodiment, the medicament comprises a TRPV1 antagonist formulated together with a LPAr antagonist for administration by inhalation.

In an embodiment, the medicament is in the form of a kit comprising a TRPV1 antagonist and an LPAr antagonist which are separately formulated. In an embodiment, the medicament is in the form of a kit comprising a TRPV1 antagonist and an LPAr antagonist which are separately formulated for administration at the same time. In an embodiment, the medicament is in the form of a kit comprising a TRPV1 antagonist and an LPAr antagonist which are separately formulated for administration by injection at the same time. The term at the same time refers to administration by any suitable means within 24 hours. Administration at the same time preferably refers to administration any suitable means within 12 hours. Administration at the same time more preferably refers to administration any suitable means within 6 hours. Administration at the same time yet more preferably refers to administration any suitable means within 2 hours. Administration at the same time most preferably refers to administration by any suitable means within 1 hour. Administration at the same time can refer to administration by any suitable means within 30 minutes. In an embodiment, the medicament is in the form of a kit comprising a TRPV1 antagonist and an LPAr antagonist which are separately formulated for administration by injection. In an embodiment, the medicament is in the form of a kit comprising a TRPV1 antagonist and an LPAr antagonist which are separately formulated for administration by separate injection at the same time. In an embodiment, the medicament is in the form of a kit comprising a TRPV1 antagonist and an LPAr antagonist which are separately formulated for administration by inhalation and/or injection. In an embodiment, the medicament is in the form of a kit comprising a TRPV1 antagonist and an LPAr antagonist which are separately formulated for administration by inhalation. Certain kits herein comprise two or more pharmaceutically active ingredients which are separately packaged for use together. Kits further optionally comprise one or more devices for administration of the one or more active ingredients. The one or more active ingredients are optionally packaged within the one or more devices. Exemplary devices include one or more syringe or one or more inhaler device.

In a preferred embodiment, the methods herein comprise administration of a combination of a TRPV1 antagonist and an LPAr antagonist. In a preferred embodiment, the methods herein comprise administration of a combination of a TRPV1 antagonist and an LPAr antagonist at the same time. In a preferred embodiment, the methods herein comprise administration of a combination of a TRPV1 antagonist and an LPAr antagonist which are formulated together for administration at the same time. In an embodiment, formulation is for administration by injection. Injection includes, among others, intravenous injection, intramuscular injection and intraperitienal injection. In an embodiment, administration is by inhalation. In specific embodiments of methods herein, the TRPV1 antagonist and the LPAr antagonist are formulated separately for administration at the same time.

In specific embodiments, a pharmaceutical composition is provided which comprises one or more TRPV1 antagonist, one or more LPAr antagonist or a combination of one or more TRPV1 antagonist and one or more LPAr antagonist and optionally a pharmaceutically acceptable carrier for use in the treatment of asthma or more specifically in the treatment of acute asthma, or an asthma attack. In an embodiment, such pharmaceutical compositions comprise a combination of one or more TRPV1 antagonist and one or more LPAr antagonist. In an embodiment, such pharmaceutical compositions comprise a combination of one TRPV1 antagonist and one LPAr antagonist. In an embodiment, in such pharmaceutical compositions the molar ratio of TRPV1 antagonist to LPAr antagonist in the composition ranges from 50:1 to 1:50. In an embodiment, in such pharmaceutical compositions the molar ratio of TRPV1 antagonist to LPAr antagonist in the composition ranges from 10:1 to 1:10. In an embodiment, such pharmaceutical compositions comprise a pharmaceutically acceptable carrier. In an embodiment, such pharmaceutical compositions comprise a pharmaceutically acceptable carrier suitable for administration by injection. In an embodiment, such pharmaceutical compositions comprise a pharmaceutically acceptable carrier suitable for administration by inhalation.

In specific embodiments, a pharmaceutical composition is provided which comprises one or more TRPV1 antagonist, one or more LPAr antagonist or a combination of one or more TRPV1 antagonist and one or more LPAr antagonist and optionally a pharmaceutically acceptable carrier for use in the prevention or treatment of an asthma attack. In an embodiment, such pharmaceutical compositions comprise a combination of one or more TRPV1 antagonist and one or more LPAr antagonist. In an embodiment, such pharmaceutical compositions comprise a combination of one TRPV1 antagonist and one LPAr antagonist. In an embodiment, in such pharmaceutical compositions the molar ratio of TRPV1 antagonist to LPAr antagonist in the composition ranges from 50:1 to 1:50. In an embodiment, in such pharmaceutical compositions the molar ratio of TRPV1 antagonist to LPAr antagonist in the composition ranges from 10:1 to 1:10. In an embodiment, such pharmaceutical compositions comprise a pharmaceutically acceptable carrier. In an embodiment, such pharmaceutical compositions comprise a pharmaceutically acceptable carrier suitable for administration by injection. In an embodiment, such pharmaceutical compositions comprise a pharmaceutically acceptable carrier suitable for administration by inhalation.

In a specific embodiment, a kit for treating asthma is provided which comprises one or more TRPV1 antagonist and one or more LPAr antagonist separately packaged for use together. In an embodiment of such a kit, the one or more TRPV1 antagonist and one or more LPAr antagonist are separately formulated for administration at the same time. In a specific embodiment, a kit for prevent an asthma attack or treating an asthma attack is provided which comprises one or more TRPV1 antagonist and one or more LPAr antagonist separately packaged for use together. In an embodiment of such a kit, the one or more TRPV1 antagonist and one or more LPAr antagonist are separately formulated for administration at the same time.

In embodiments, the pharmaceutical compositions, medicaments and kits herein optionally comprise additional active ingredients that are useful in addition for treating asthma or asthma attacks or are useful for treating allergic reactions. Optional additional active ingredients include, among others, one or more of the following: short-term β-agonists, long-term β-agonists, methylxanthines, anticholinergic agents, antihistamines, corticosteroids, leukotriene antagonists, decongestants, or non-steroidal anti-inflammatory agents.

Methods for making a medicament for the treatment of asthma or the prevention of or treatment of an asthma attack, which comprises combining one or more TPRPV1 antagonists and/or one or more LPAr antagonists with a pharmaceutically acceptable carrier, is provided. In an embodiment of such methods, one or more TPRPV1 antagonists and one or more LPAr antagonists are combined with a pharmaceutically acceptable carrier. In an embodiment of such method, the medicament comprises one or more TPRPV1 antagonists and one or more LPAr which are separately packaged in a kit for use together. In an embodiment of such method, the medicament comprises one or more TPRPV1 antagonists and one or more LPAr which are separately formulated for use together.

The disclosure provides use of a TRPV1 antagonist, an LPAr antagonist or both for the treatment of asthma or the prevention of asthma attacks. In a specific embodiment, use of the TRPV1 antagonist, the LPAr antagonist or both is for treatment of an asthma attack. In a specific embodiment, the disclosure provides use of a combination of a TRPV1 antagonist and an LPAr antagonist for the treatment of asthma. In a specific embodiment, the disclosure provides use of a combination of a TRPV1 antagonist and an LPAr antagonist for the prevention or treatment of an asthma attack. In such uses, the TRPV1 antagonist, the LPAr antagonist or both are formulated for administration by injection. In such uses, the TRPV1 antagonist, the LPAr antagonist or both are formulated for administration by inhalation.

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group is disclosed herein, it is understood that all individual members of the group and all subgroups, including any isomers and enantiomers of the group members, and classes of compounds that can be formed using the substituents are disclosed separately. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

One of ordinary skill in the art will appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

### THE EXAMPLES

A novel lung-carotid body-lung reflex pathway has been demonstrated in acute allergen/bradykinin-evoked bronchoconstriction and respiratory disturbances in an animal model of asthma. The examples demonstrate that blocking LPA signaling ameliorates bradykinin-invoked bronchoconstriction in anesthetized asthmatic animals. Further, in conscious animals, where the severity of respiratory disturbances are fully exhibited without the mitigating effects of anesthesia, the treatment remains effective. The results presented in these examples provide a mechanistic explanation for the importance of the carotid body in acute asthmatic symptoms and demonstrates therapeutic drug targets for emergency intervention.

U.S. provisional application 62/534,638, filed Jul. 19, 2017 is incorporated by reference herein in its entirety. More specifically, figures and examples therein are incorporated by reference herein.

### Example 1: LPA Activates TRPV1 and LPA-Specific Receptors in the Carotid Body

LPA activates TRPV1 receptors^{71,72}. TRPV1 receptors are expressed in the terminals of petrosal neurons that innervate the carotid body (i.e., the axon terminals of chemosensory afferent⁴¹). LPA also binds to 6 LPA-specific G-protein coupled receptors (GPCR), LPAr 1 through 6. RT-PCR was used to test for the presence of these receptors in the carotid body. cDNA for LPAr 1, 3, 4, and 6 were found in the carotid body. cDNA for LPAr 3 and 6 were found in petrosal ganglia. cDNA for LPAr 1, 3, 4 and 6 were found in the superior cervical ganglia. No evidence was found for expression of LPAr 2 in these carotid body associated tissues (data not shown).

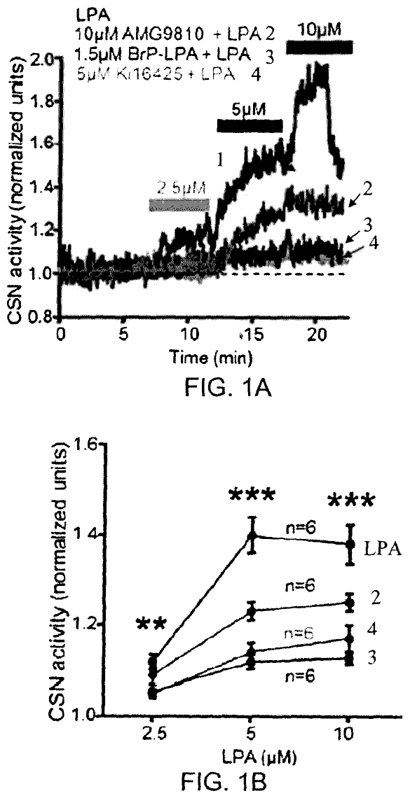

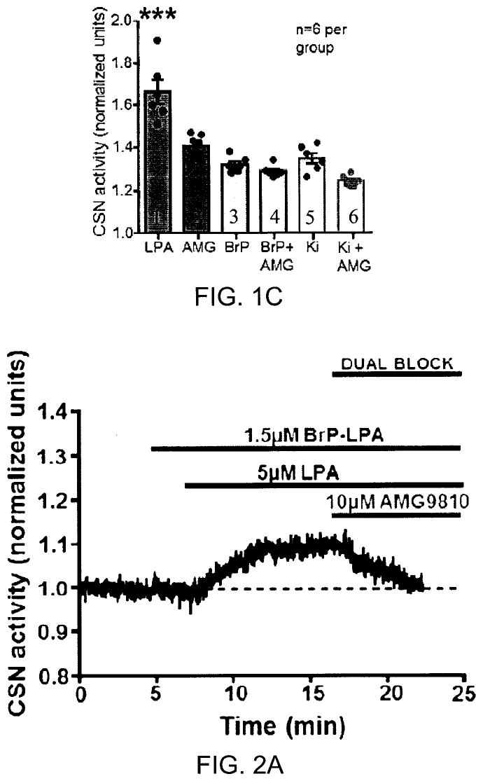

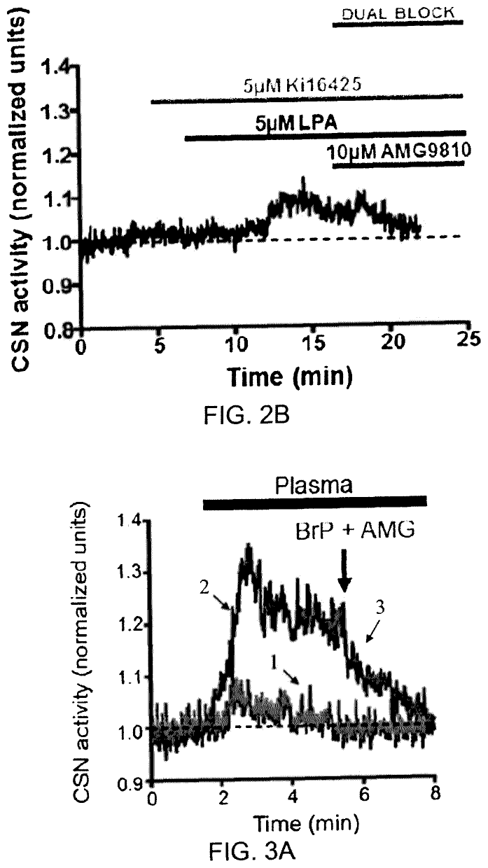

The functional effects of LPA (18:1 unless otherwise stated) on glomus cells were assessed using Fura 2 calcium imaging. LPA (5 μM) increased intracellular calcium release producing calcium spikes of similar order of magnitude to that produced by 20 mM potassium (data not shown). LPA species present in blood (LPA (16:0), LPA (18:1) and LPA (18:2)) were shown to have functional effects on the carotid body output using an en bloc perfused carotid body preparation (data not shown^{41,89}). The LPA species tested exhibited a dose dependent effect on carotid sinus nerve activity (data not shown). To determine which receptors mediate this, the effects of LPAr blockade (BrP-LPA, 1.5 μM or Ki16425, 5 μM), TRPV1 blockade (AMG9810, 10 μM) and dual blockade (FIGS. 1A-C, FIGS. 2A-B) were assessed. AMG9810 reduced 5 μM LPA-mediated carotid sinus nerve excitation by 41±6%; BrP-LPA or Ki16425 reduced 5 μM LPA-mediated excitation by 70±3% or 61±9%, respectively; and dual blockade with AMG9810 and either BrP-LPA or Ki16425 reduced excitation by 77±5% or 89±3%, respectively. These data demonstrate LPA stimulation of the carotid body involves both TRPV1 and LPA-specific GPCR's.

### Example 2: LPA Activation of the Carotid Body Causes Vagal Efferent Activity

To determine if LPA activation of the carotid body stimulates chemoafferents (not baro-afferents) and, is capable of inducing parasympathetic (vagal) activity with the capacity to cause bronchoconstriction, an in situ dual-perfused preparation as described below was used. This preparation allows artificial perfusion of the carotid bodies and brainstem independently, while recording from phrenic and vagal efferents⁹⁰. Once baseline conditions were established (brainstem perfused with 40 Torr PCO₂, balanced with O₂; carotid bodies perfused with 35 Torr PCO₂ and 100 Torr PO₂, balanced with N₂), carotid body viability was tested using a 10 min bout of hypoxic perfusate (50 Torr PO₂ and 40 Torr PCO₂ balanced with N₂). Following recovery, the brainstem perfusate was switched to hypocapnia (PCO₂≥20 Torr, balance O₂). Central hypocapnia induces a state of complete apnea (cessation of phrenic bursts >1 min) and optimizes the sensitivity of the preparation to carotid body stimuli⁹¹. Next, a bolus of 5 μM LPA was delivered to the carotid body circulation. With carotid bodies intact, hypoxia and LPA induced increases in phrenic (indicating LPA recruits carotid body chemoafferents) and vagal nerve efferent discharge (data not shown). With the carotid sinus nerve resected, the response to hypoxia and LPA was abolished (data not shown).

### Example 3: LPA Activation of the Carotid Body Causes Acute Bronchoconstriction

To determine if LPA activation of the carotid body is capable of causing acute bronchoconstriction in vivo, lung function measurement were performed in naïve animals using an anesthetized artificially ventilated preparation with the Flexivent respirator system. Saline, 5 μM LPA and 6 μM NaCN (an independent test of carotid body sensory viability³⁸), were delivered consecutively to each animal (0.5 ml bolus injected over 1 min via vena cava catheter; 10 min between challenges). LPA and NaCN, but not saline, induced increased airway resistance in preparations with intact CSN (data not shown). These challenges had no effect in preparations with bilateral carotid sinus nerve resection, demonstrating the necessity of the carotid body in LPA-induced bronchoconstriction.

### Example 4: Acute Asthmatic Bronchoconstriction is Associated with an Increase in Arterial LPA

If asthma-associated acute bronchoconstriction is caused by activation of the carotid bodies by LPA released from an allergen-challenged asthmatic lung, (a) the arterial concentration of LPA should increase significantly during acute inflammation and (b) the plasma from asthmatic rats should stimulate an isolated carotid body in a TRPV1 and LPA receptor dependent fashion. To assess this, an ovalbumin-sensitized (OVA) Brown Norway rat model of asthma⁹², which exhibits many of the salient features of human asthma, was used. This model exhibits an increased Inflammatory Index Score (i.e., elevated eosinophil cell counts in airways and bronchoalveolar lavage fluid, increased airway smooth muscle thickening and epithelial goblet cell metaplasia, increased baseline airway resistance and increased gene expression of chemoattractant in lung tissue. While methacholine is often used to trigger robust acute bronchoconstriction in this model, bradykinin was used in these experiments because parasympathetic efferents are cholinergic, and thus exogenous methacholine is likely to mask parasympathetic involvement. Bradykinin delivered on day 28 (0.4 mg; nebulized) caused a marked increase in airway resistance in OVA but not naïve rats within 20 min. Arterial plasma samples were analyzed with ELISA prior to and 5 min following bradykinin stimulation showing that bradykinin had no effect on plasma concentration of LPA in naïve rats, but significantly increased LPA in the OVA group. The coefficient of variation for all duplicate samples was 2.4±0.3%.

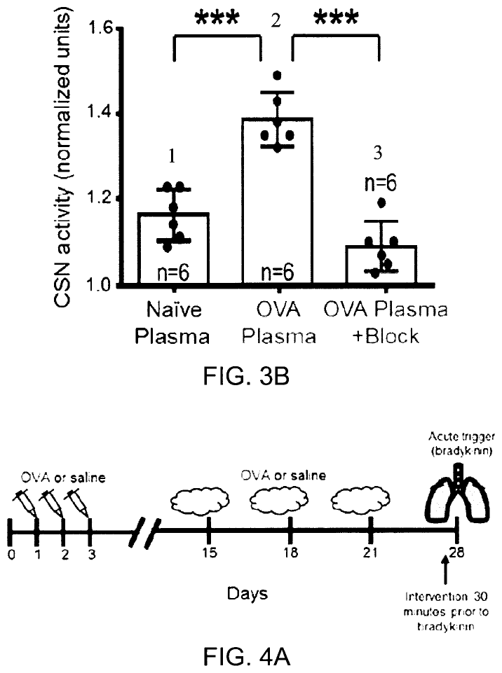

To test if the LPA in plasma from asthmatic rats is sufficient to stimulate the carotid body, plasma was harvested from naïve and asthmatic animals 3 hrs after allergen challenge and the effects of harvested plasma on carotid sinus nerve activity in the isolated en bloc carotid body preparation was measured. Naïve and asthma plasma caused 16±2% and 39±3% increases in carotid sinus nerve activity, respectively. Dual blockade of TRPV1 and LPA receptors with AMG9810 and Brp-LPA reduced the response to asthma plasma by 79±2% (FIG. 3A-3B).

### Example 5: Neuronal Pathway Involving LPA Signaling is Required for Acute Asthmatic Bronchoconstriction

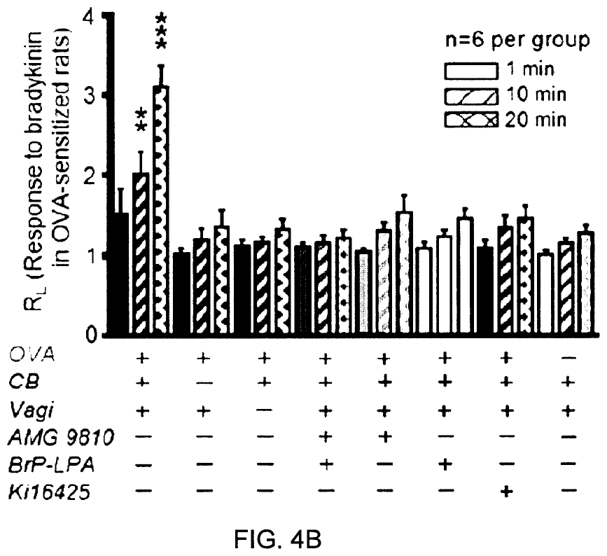

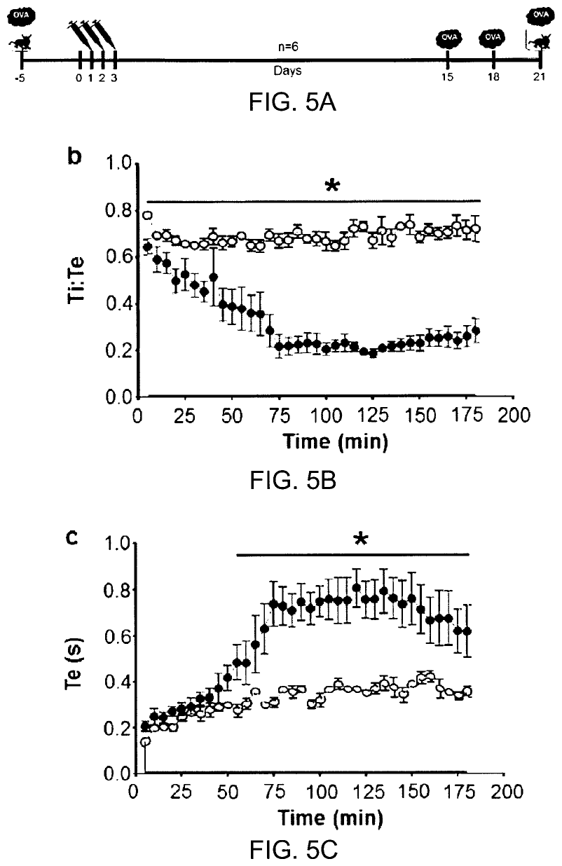

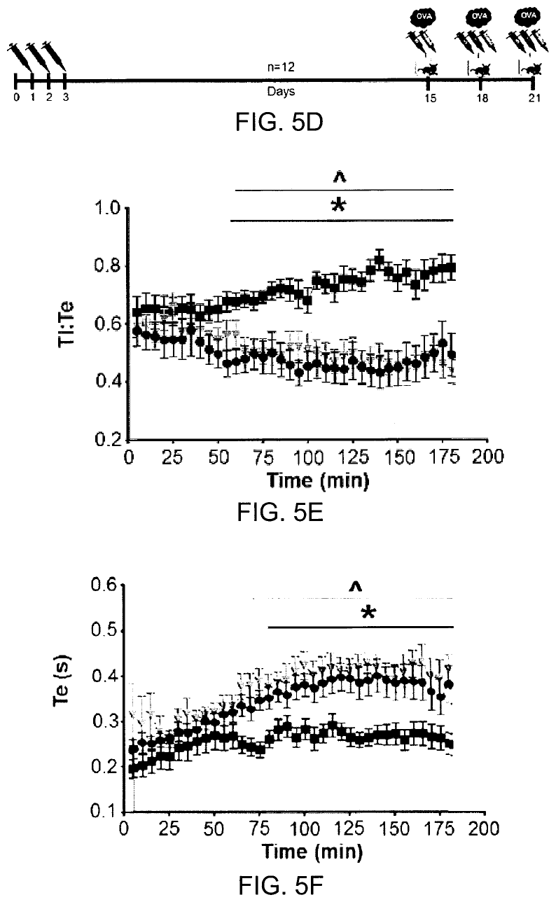

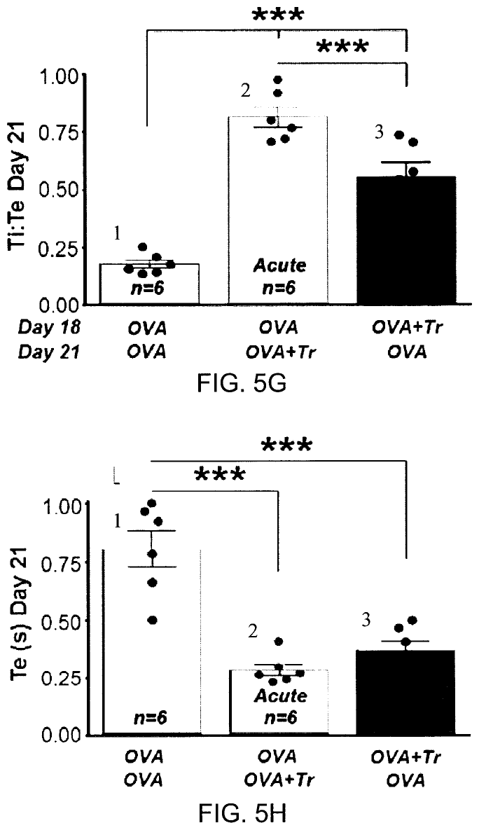

In order to examine the involvement of LPA-induced carotid body activity on airway resistance in response to bradykinin nebulization, alfaxan-anesthetized animals and the Flexivent system were used. These experiments were performed with a number of acute interventions affecting the proposed pathway, including vagotomy, carotid body denervation, and TRPV1 and/or LPA receptor blockade. Prior to bradykinin, these interventions had no effect on airway resistance (Holm-S̆idák post hoc test comparing asthma control vs any manipulation group: p>0.06) and as expected, bradykinin had no effect on non-asthmatic lungs (p>0.3). However, the acute bronchoconstriction induced by bradykinin in asthmatic lungs was diminished by at least 60% with all interventions (FIG. 4B). The loss of bradykinin-induced bronchoconstriction also occurred in chronic carotid body denervated, but not sham rats (data not shown).

To ensure maintenance of vagal-vagal reflexes after carotid body denervation and thereby rule out the possibility of indirect effects of carotid body denervation, it was tested whether carotid body denervation abolished bronchoconstriction induced by aerosolized capsaicin (a potent activator of C-fibre-mediated vagal-vagal reflexes). Capsaicin-induced bronchoconstriction was abolished by vagotomy, but not affected by carotid body denervation. Similar results were obtained in naïve and asthmatic animals. In addition, to ensure that increased plasma LPA was not provoking increased airway resistance via a vagal-vagal reflex, asthmatic rats were exposed to nebulized LPA. LPA had no immediate effect on airway resistance. An increase in airway resistance occurred after 30 min, but this was abolished by carotid body denervation. Together, these data indicate that the increase in lung resistance with nebulized LPA is dependent on the carotid body.

### Example 6: Blocking LPA Signaling Reduces Acute Bronchoconstriction Following Allergen Challenge

In order to determine if blocking LPA signalling can be used to limit the severity of acute allergen-induced respiratory distress in conscious animals, 12 OVA rats were exposed to an aerosol containing ovalbumin (150 mg, nebulized) to trigger acute asthmatic bronchoconstriction and 20 min later, injected with a cocktail of TRPV1 and LPAr (ip 10 μM/kg AMG9810 and 3 mg/kg BrP-LPA, in 0.5 ml, FIG. 6d) antagonists. After the injection, animals were placed in a whole-body plethysmograph to monitor breathing. Following ovalbumin, expiratory time (Te) increased and inspiratory time: expiratory time (Ti:Te) ratio decreased, indicative of expiratory difficulty associated with acute asthmatic bronchoconstriction (FIG. 6a-f⁹³). However, the increase in Te and decrease in Ti:Te after 100 min was reduced in dual-block treated animals (Ti:Te: −28.3±3.4%; Te: −72.9±2.3%) compared to vehicle-injected or sham groups (Ti:Te: −49.2±4.8%; Te: −59.1±3.1% and, Ti:Te: −55.0±4.9%; Te: −62.7±2.9%; Ti:Te: F2,35=10.064, p<0.001; Te: F_{2,35}=6.495, p=0.004, FIG. 6e, f), indicating that this treatment can be used to reduce the severity of acute allergen-induced respiratory disturbances. Furthermore, treatment given 3 days prior to a final ovalbumin challenge was also mitigating, suggesting a long-term beneficial effect of this treatment (FIG. 6g, h).

### Example 7: Essential Role for the Carotid Body in Asthma

The carotid bodies have been implicated in asthma^{94,95,96} but a role for the carotid bodies is unsupported by clinical trials⁴⁶ and bilateral carotid body resection in humans is not advised because it abolishes the hypoxic ventilatory response. Nonetheless, activating the carotid bodies causes bronchoconstriction in normal lungs^{28-30, 32-35} and in one study in an animal model of asthma²⁷, carotid body denervation is reported to reduce the severity of nebulized methacholine induced hyper-responsiveness. While recent focus on the carotid bodies has been related to their role in exciting sympathetic activation, expected to cause bronchodilation via beta-receptor activation, as described herein the dual-perfused in situ preparation⁹¹ was used to show that carotid body stimulation also causes an increase in vagal (presumably parasympathetic and acetyl-cholinergic) activity likely capable of causing bronchoconstriction. Nebulized methacholine, used as the provocation in most animal studies of asthma, is likely to short circuit this parasympathetic pathway, possibly explaining why its importance has been underestimated in asthma. In the studies described herein, acute asthmatic symptoms were induced with allergen or bradykinin; both of which only cause pronounced bronchoconstriction in asthmatic lungs.

Bradykinin produced a ˜3-fold increase in lung resistance in asthmatic (but not naïve) rats that was ameliorated by carotid body denervation and/or vagotomy. It was also shown that bradykinin and allergen increase plasma LPA in asthmatic (but not naïve) rats and that this plasma is capable of stimulating an isolated carotid body via an LPA receptor-dependent mechanism. These data demonstrate the existence of a carotid body-mediated vagal reflex capable of increasing vagal efferent activity causing bronchoconstriction in response to LPA. As the carotid bodies are a main driver of ventilation, a role in mediating bronchoconstriction appears counterintuitive, however this mechanism may be responsible for maintaining rigidity of airways during increased ventilation and cough, and/or minimizing dead-space ventilation^{97,98}. LPA is a trigger of carotid body activity in acute asthma

The data herein demonstrate a role for a carotid body-vagal reflex in an animal model of asthma. Arterial hypoxemia caused by poor lung function during an asthmatic attack was suspected as a trigger of the carotid body in asthma⁹⁹¹⁰⁰¹⁰¹⁵²⁻⁵⁴. Indeed, acute bronchoconstriction caused by bradykinin resulted in hypoxemia in the OVA model. However, in asthmatic humans, the severity of respiratory distress does not always correlate with arterial hypoxemia^{10210355,56,} and others have demonstrated no bronchoconstriction with reduced inspired O₂^{50,104 57}, suggesting additional and/or different carotid body triggering mechanisms.

Notwithstanding a possible role for hypoxia, these data demonstrate that the carotid body is activated by μM concentrations of LPA. LPA is a central player in allergen induced asthmatic lung inflammation^{10510658,59.} During allergen challenge, several species of LPA are released by lung epithelial cells into the surrounding tissue^{73,10760} and regulate prostaglandin levels, expression of Th2 cytokine receptors and IL13 signal transduction¹⁰⁸⁶¹. LPA may also increase the sensitivity of airway smooth muscle independently¹⁰⁹⁶² and has been shown to augment acetylcholine mediated airway smooth muscle contractility¹¹⁰⁶³. However, LPA is also released systemically, in arterial plasma^{73, 106-108}. In the OVA model, LPA in arterial plasma (measured by ELISA) increases from ˜4 μM to 8 μM following allergen/bradykinin. In the en bloc preparation, μM concentrations of exogenous LPA caused increased carotid sinus nerve activity; and in the dual perfused in situ and naïve in vivo preparations, LPA caused increased vagal activity and bronchoconstriction, respectively, both of which were abolished by carotid body denervation. Carotid sinus nerve activity was heightened in the en bloc carotid body preparation when plasma collected from asthmatic rats following OVA challenge was delivered into the preparation. This effect was abrogated by TRPV1 and LPA receptor blockade and not demonstrated with plasma drawn from naïve rats. Furthermore, blocking LPA signaling in the OVA model reduced bronchoconstriction (in anesthetized preparations) and ventilatory effects associated with respiratory difficulty (in conscious animals) following allergen provocation. Together, these data demonstrate that LPA released systemically by an allergen/bradykinin-provoked lung is likely a trigger of carotid body activity.

Approximately 70% of the en bloc carotid body's response to LPA was blocked by BrP-LPA (an LPAr 1-4 antagonist) and Ki16425 (a LPAr 1,3 and weak LPAr2 antagonist). The carotid bodies contain LPAr 1, 3, 4, and 6 and the petrosal ganglia, containing cell bodies of carotid body chemosensory afferents, contain LPAr 3 and 6. Recent studies suggest LPA also activates TRPV1 both directly by binding to the C-terminus of TRPV1⁷¹ and indirectly via LPAr activation and PKCε phosphorylation⁷². As TRPV1 is expressed in chemosensory afferents⁴¹, and the TRPV1 antagonist AMG9810 blocks the LPA response of the en bloc preparation that remains after BrP-LPA or Ki16425 blockade, TRPV1 activation must also be part of the trigger mechanism. TRPV1 is activated by several other inflammatory mediators that have demonstrated effects on the carotid body, including IL-6^{111,112} prostaglandins, and TNFα¹¹³. These inflammatory mediators are also released by the lung during an acute asthmatic attack^{73,108}, but it is presently unknown if they act as additional triggers for carotid body-mediated bronchoconstriction.

### Example 8: LPA Signaling at the Carotid Body Provides New Targets for Pharmacological Intervention

Although using an in vivo anesthetized ventilated preparation allows the ability to measure lung mechanics directly, monitoring asthmatic responses over extended periods with this method is limited due to the accumulating risk of lung injury. In experiments described herein, dual blockade following allergen challenge was delivered in the ovalbumin rat model in order to assess the efficacy of combined LPAr+TRPV1 inhibition as a method of therapy. Behavioral indices of airway resistance (Ti:Te) were reduced ˜60% by combined blockade indicating that the bulk of neurogenic bronchoconstriction in the ovalbumin rat model of asthma is dependent on the LPAr+TRPV1 pathway. The protocol used a randomized within-subject design allowing the effects of dual treatment, saline and vehicle to be tested and compared in the same rat. Remarkably, it was found that the subset of animals receiving OVA+dual blockade on day 18 and receiving OVA+saline on day 21, had lower indices of airway resistance on day 21 than animals receiving OVA+saline on both days. The combined blockade of LPAr+TRPV1 as an asthmatic treatment is therefore of particular interest because it reduces airway resistance even when administered following allergic induction and had beneficial effects lasting several days. It is also noteworthy that much of the efficacy of the dual blockade in relieving asthmatic symptoms was preserved when ovalbumin was used in conscious ovalbumin-sensitized animals.

### Example 9: Materials and Methods

### Animals

Male Brown Norway (BN/Crl, p28-35, 80-150 g) and Sprague Dawley rats (p21-28, 50-80 g) rats were purchased from Charles River (QC). Where appropriate, the use of rat strain is described in the experimental procedures below. Experimental procedures were approved by the University of Calgary Animal Care and Use Committee. Animals were housed in pairs in a 12 h light/dark cycle with water and chow freely available.

### Chemicals and Reagents

Oleoyl-Lysophosphatidic acid (18:1 LPA), BrP-LPA and AMG9810 were purchased from Cayman Chemical (Cayman, Ann Arbor, Mich.). D-(+)-sn-1-O-linoleoyl-glyceryl-3-phosphate (18:2 Linoleoyl LPA) was purchased from Echelon Biosciences (Salt Lake City, Utah). 1-palmitoyl-2-hydroxy-sn-glycero-3-phosphate (16:0 Lysophosphatidic acid) was purchased from Avanti Polar Lipids (Alabaster, Ala.). FURA-2AM was purchased from Invitrogen (Carlsbad, Calif.). Ovalbumin, pertussis toxin, aluminum hydroxide, MgSO₄, NaH₂PO₄, KCl, NaHCO₃, NaCl, glucose, sucrose, CaCl₂, tween 80, dimethyl sulfoxide, pancuronium bromide, sodium cyanide, trypsin, Ham's F12, Ki16425 and ethanol were purchased from Sigma-Aldrich (Sigma-Aldrich, Oakville ON).

### Statistics and Analysis

All statistical analysis was performed in SigmaPlot Vs 13.0 (Systat Software San Jose, Calif.). Tests used are provided at the end of each specified experiment section. Normally-distributed data were analyzed using parametric statistics and presented as mean±sem; the Holm-S̆idá\ddot{k} Post hoc test was used for pairwise multi-comparisons of normal data, unless otherwise stated. All other data were analyzed using non-parametric statistics and presented as median±range.

### RT-PCR

Carotid bodies, petrosal and superior cervical ganglia were collected from n=6 Sprague Dawley rats and tissue were stored in RNALater (Sigma-Aldrich, St. Catherines, QC) at 4° C. until analysis. Total RNA extracted from isolated carotid bodies and superior cervical and petrosal ganglia was purified with the RNeasy Mini kit (Qiagen, Germantown, Md.) per manufacturer's instructions. Total RNA (200 ng) from each sample was converted to single-stranded cDNA using the Quantiect RT-PCR (Qiagen, Germantown, Md.) with random primers 10 μM. PCR amplification was carried with a 20p1 reaction volume containing 1 μl of a cDNA, 7 μl ddH₂O, primers at 1 μL each, and 10 μL PCR enzyme. PCR was performed under the following conditions: 95° C. for 3 min followed by 45 cycles of denaturation (95° C. for 30 s), annealing (60° C. for 30 s), and elongation (72° C. for 1 min), followed by 3 min at 72° C. before refrigeration (4° C.). Suitable primer sequences (sense and antisense) for LPAr1-LPAr6 and hypoxanthine phosphoribosyltransferase were employed. The amount of cDNA for each tissue was confirmed with a NanoDrop spectrophotometer (Thermo Scientific, Burlington Ontario). Changing the lane of the ladder identified which tissue was run on which gel. The PCR products were analyzed by electrophoresis using a 1% agarose gel and visualized under UV light with BioRad Image Lab 3.0 Software (Missisauga ON).

### Carotid Body Type I Cell Isolation and Calcium Imaging

Sprague-Dawley rats (80-150 g, P21-28) were anaesthetised with isoflurane (3-5% in O₂) and carotid bodies were harvested and digested in 0.4 mg ml⁻¹ collagenase type I (Worthington Biochemical Corporation, Lakewood, N.J.) and 0.2 mg ml⁻¹ trypsin type I (Sigma-Aldrich, St. Catherines, QC) in DPBS enzyme solution with low CaCl₂) (86 μM) and MgCl₂ (350 μM), for 20 min at 37° C. followed by dissociation with forceps and incubated for an additional 7 min. The tissue was centrifuged (115 g) for 3 min, supernatant removed and the pellet re-suspended in Ham's F12 (Sigma-Aldrich, St. Catherines, QC) supplemented with 10% heat inactivated fetal bovine serum (Biowest, San Marcos, Tex.). Cells were released by trituration with fire polished silanised Pasteur pipettes (Sigma-Aldrich, St. Catherines, QC). Type I cells were plated on 15 mm round poly-d-lysine-coated (0.1 mg ml⁻¹) glass coverslips (Warner Instruments, Hamden, Conn.) and incubated at 37° C. in 5% CO₂, 10% O₂ ˜2 hours before use; cells were used for experiments within 8 hours of isolation.

Type I cells were loaded with 5 μM FURA-2AM (Invitrogen, Carlsbad Calif.) in serum free Ham's F12 nutrient media for 30 min at room temperature in humidified 5% CO₂, 10% O₂, before being transferred to FURA-2AM-free media in the same conditions for 20 min. Coverslips were placed in an RC-25F (Warner Instruments, Hamden, Conn.) 500 μl recording chamber at 34-36° C. Image acquisition was controlled by Metafluor software (Molecular Devices, Sunnyvale Calif.) and cells were visualised using a Nikon TE2000-U inverted microscope with a CFI super fluor ×40 oil immersion objective. The FURA-2 loaded cells were excited by 50 ms exposures to 340/380 nm light using a Lambda 10-3 filter wheel every 5 s and emitted light was recorded at 510 nm using a Coolsnap HQ2 CCD camera (Photometrics, Tucson Ariz.).

Cells were continuously perfused with a standard HEPES buffered salt solution containing: 140 mM NaCl, 4.5 mM KCl, 2.5 mM CaCl₂, 1 mM MgCl₂, 11 mM glucose, 10 mM HEPES, adjusted to pH 7.57 with NaOH at room temperature to yield a pH of 7.4 at 37° C. Solution containing LPA (5 μM, 300 seconds) or high potassium (20 mM, 70 seconds) was switched from independent reservoirs and superfused over the cover slips. The change in fluorescence ratio (F₃₄₀/F₃₈₀) from baseline to peak (ΔF₃₄₀/F₃₈₀) was measured for each challenge. Significance was tested using one-way ANOVA of peak emission against baseline values.

### Broncho-Alveolar Lavage Fluid

Bronchoalveolar lavage fluid was collected from n=7 OVA and n=7 Naïve rats following challenge with bradykinin (below). With the upper trachea cannulated, lungs were lavaged (10 ml/lavage) with saline (0.9%). Cells in bronchoalveolar were sedimented by centrifugation (20 min at 4500 g, 4° C.) and resuspended in phosphate buffered saline. 100 ml of bronchoalveolar lavage fluid was centrifuged (Shandon Cytospin 4 cytocentrifuge, Thermo Scientific, Waltham, Mass., 6 min at 4500 g) and cells collected on non-coated glass slides, fixed in 95% ethanol and stained with hematoxylin and eosin. Total leukocytes were determined by hemacytometer counting. Differentiation of 200 cells was completed according to standard morphologic criteria^{114,116}. Samples were compared with Student's unpaired two-sided t-test.

### Lung Histology, Immunohistochemistry, and Gene Expression

Following exposure to bradykinin (below) both lungs were inflated with formalin (10-15 ml, ˜30 mmHg) in n=7 OVA and n=7 Naïve rats, rapidly excised then fixed in formalin. The left lung was hemisected at the level of the bronchus according to previously described methods¹¹⁶, embedded in paraffin and subsequently 4 μm thick sections cut and de-paraffinized. To determine goblet cell metaplasia, sections were stained with periodic acid and schiffs reagent and counter-stained with hematoxylin. The numbers of goblet cells were expressed as cells per circumference of bronchial epithelium. Immunohistochemistry was perfirned for smooth muscle actin, using DAB as chromogen. Smooth muscle hyperplasia was quantified as the intensity of stain per area of section using Image J (NIH, Bethesda ML). Total inflammation score was evaluated by a blinded observer (MMK) using a semi-quantitative scoring system to evaluate the fraction of the airway that was occupied by inflammatory cell infiltrates: 4=robust inflammation (more than 50% of airway circumference surrounded by inflammatory cell infiltrates); 3=moderate inflammation (25-50% of airway circumference surrounded by inflammatory cell infiltrates); 2=mild inflammation (10-25% of airway circumference surrounded by inflammatory cell infiltrates); 1=minimal inflammation (<10% of airway circumference surrounded by inflammatory cell infiltrates) and a score of 0=no inflammatory cell infiltrates; for lung parenchyma the same system was used but in reference to the percent of alveoli involved by inflammation.

Gene expression of IL4 and eotaxin (CCL11) were probed with SYBR green qPCR in relation to β-actin. Total RNA extracted from left lungs was purified with the RNeasy Mini kit (Qiagen, Germantown, Md.) per manufacturer's instructions. The amount of cDNA for each tissue was confirmed with a NanoDrop spectrophotometer (Thermo Scientific, Burlington Ontario). Total RNA (1000 ng) from each sample was converted to single-stranded cDNA using the Quantiect RT-PCR kit (Qiagen, Germantown, Md.) with 10 μM of random primers. PCR amplification was carried with a 20p1 reaction volume containing 1p1 of a cDNA, 7p1 ddH₂O, primers at 1 μL each, and 10 μL SYBR green (QuantiNova Qiagen, Germantown, Md.). PCR was performed under the following conditions: 95° C. for 3 min followed by 45 cycles of denaturation (95° C. for 30 s), annealing (60° C. for 30 s), and elongation (72° C. for 1 min), followed by 3 min at 72° C. before refrigeration (4° C.) with melt curves produced for all samples; samples for each rat and gene were run in triplicate with the Eppendorf Mastercycler (Eppendorf, Mississauga ON). Suitable primer sequences for IL4, CCL11, and β-actin were used. Data are expressed as cycles threshold and 2^{ΔCTT} was calculated by standard methods¹¹⁷ Data were compared with Student's unpaired two-sided t-test or Mann Whitney Rank Sum Test (inflammation score).

### LPA Concentration

Arterial blood samples (0.7 ml) were drawn prior to and following bradykinin challenge in anesthetized experiments. Blood samples were spun for 20 min at 4000 g in heparinized (5 iu) tubes at room temperature, plasma was drawn and snap frozen and stored at −80° C. until analysis (no sample experienced freeze thaw cycles). Plasma samples were tested for LPA concentration by the K-2800S ELISA plate (LPath Inc, San Diego, Calif.; Echelon Biosciences, Salt Lake City, Utah) in strict accordance with manufacturers specifications. Samples were run in duplicate as per manufacturers recommendations and coefficient of variation calculated. Samples were analyzed by two-way repeated measures ANOVA.

In order to verify the standard curve under experimentally appropriate conditions 1) the curve was compared with a plasma sample spiked with known LPA 18:1 concentrations; 2) Plasma preparation with EDTA and Heparin were compared and; 3) Venous vs arterial samples were compared. Data were analyzed with Pearson correlation (standard curves) and Students' independent t-test.

### Example 10: En Bloc Perfused Carotid Body Preparation

Sprague-Dawley rats were anesthetized with isoflurane and then decapitated, the carotid bifurcation, including the carotid body, carotid sinus nerve, and superior cervical ganglion, was quickly removed and transferred to a beaker (100 ml) containing carbogen (95% O₂, 5% CO₂) equilibrated physiological saline (1 mM MgSO₄, 1.25 mM NaH₂PO₄, 4 mM KCl, 24 mM NaHCO₃, 115 mM NaCl, 10 mM glucose, 12 mM sucrose, and 2 mM CaCl₂)). After ˜20 min, the carotid bifurcation was transferred to a recording chamber with a built-in water-fed heating circuit (AR, custom made) and the common carotid artery was immediately cannulated for luminal perfusion with physiological saline (as above) with a peristaltic pump set at 15 ml/min to maintain a constant pressure of 100 mmHg. The perfusate was equilibrated with computer-controlled gas mixtures of 100 Torr PO₂ and 35 Torr PCO₂ balanced with N₂ and recirculated throughout the experiments (yielding pH ˜7.4) and heated to 37±0.5° C. The carotid sinus region was bisected and the carotid sinus nerve was de-sheathed. Chemosensory discharge was recorded extracellularly from the whole de-sheathed carotid sinus nerve, hooked to a platinum electrode and lifted into a thin film of paraffin oil. A reference electrode was placed close to the carotid artery bifurcation. Nerve activity was monitored using a differential AC amplifier (model 1700, AM Systems), secondary amplifier (model AM502, Tektronix, Beaverton, Oreg.), filtered (300-Hz low cut-off, 5-kHz high cut-off), displayed on an oscilloscope, rectified, integrated (200-ms time constant), and stored on a computer using an analog-to-digital data acquisition system (Digidata 1322A, Axon Instruments; Axoscope 9.0). Preparations were exposed to a brief hypoxic challenge (60 Torr PO₂) to determine viability. Preparations that failed to show a clear increase in activity during this challenge were discarded. After this challenge, preparations were left undisturbed for 30-45 min to stabilize before the experimental protocol was begun. 1) LPA was infused at three separate concentrations (2.5, 5, 10 μM) n=6 each for 18:1, 18:2 and, 16:0 species, each. 2) TRPV1 blockade (AMG9810 dissolved in dH₂O 10 μM) was infused, 5 min later LPA (18:1) was infused (2.5, 5, 10 μM, n=6). 3) LPAr blockade (BrP-LPA dissolved in DMSO 1.5 μM) was infused, 5 min later, LPA (18:1) was infused (2.5, 5, 10 μM, n=6). 4) LPAr blockade (Ki16425 dissolved in DMSO 5 μM) was infused, 5 min later, LPA (18:1) was infused (2.5, 5, 10 μM, n=6). 5) LPAr blockade (BrP-LPA dissolved in DMSO 1.5 μM) was infused, 20 min later, LPA (18:1, 5 μM) was infused, and subsequently TRPV1 blockade (AMG9810 dissolved in dH₂O 10 μM) was infused (n=6). 6) LPAr blockade (Ki16425 dissolved in DMSO 5 μM) was infused, 20 min later, LPA (18:1, 5 μM) was infused, and subsequently TRPV1 blockade (AMG9810 dissolved in dH₂O 10 μM) was infused (n=6). 7) 1 ml of plasma from naïve Brown Norway rats after saline nebulization (as per model, below) was circulated through the preparation (˜100 ml) for 10 min. 8) 1 mL of plasma drawn from ovalbumin-sensitized Brown Norway rats was drawn after ovalbumin challenge (below) and circulated through the preparation (˜100 ml), 5 min later BrP-LPA (1.5 μM)+AMG9810 (10 μM) were co-infused. Neural traces were analyzed offline using custom software (written in VEE by R. J. A. W.). One minute of carotid sinus nerve activity during each condition was rectified, summed and expressed as integrated neural discharge. The neural responses for different conditions were normalized to the baseline (normoxic) condition. LPA species differences were analyzed with two-way repeated measures ANOVA (dose×species), all other data were analyzed with one-way ANOVA for each dose or between conditions.

### Example 11: Dual Perfused Preparation

Sprague-Dawley, rats were deeply anesthetized with isoflurane via inhalation. Then, rats were cooled in 54° C. physiological saline while maintaining isoflurane anesthesia. Once respiratory movement began to subside, the rat was decerebrated at midcollicular level, transected above the renal arteries and skinned. All tissue rostral to the decerebration and all remaining cortex dorsal to the colliculi were removed. Transection and decerebration were performed in 54° C. physiological saline containing 115 mM NaCl, 24 mM NaHCO₃, 4 mM KCl, 2 mM CaCl₂, 1 mM MgSO₄, 1.25 mM NaH₂PO₄, 10 mM glucose, and 12 mM sucrose, equilibrated with 95% O₂, 5% CO₂. Once dissection was complete, the preparation was placed in a supine position in a specially designed plexiglass chamber and secured with ear bars. The descending aorta was cannulated with a double-lumen catheter. One lumen of the catheter was connected to a peristaltic pump (Gilson Minipuls 3) and used to perfuse the descending aorta in a retrograde direction with perfusate at room temperature (20° C.) and equilibrated with 40 Torr PCO₂ in O₂ (central perfusion). The other lumen was attached to a pressure transducer and used to monitor perfusion pressure. After the cannulation procedure, the speed of the peristaltic pump was increased to elevate perfusion pressure to 90 mmHg over the first few minutes with use of a custom-built computer-controlled feedback system. The common carotid arteries were tied off above the clavicles and cannulated caudal to the carotid bifurcation. A separate peristaltic pump with two channels was used to perfuse the common carotid arteries at 18.5 ml/min in order to maintain a perfusion pressure of ˜90 mmHg. Up to this point in the dissection, central and peripheral perfusions were from the same tonometer.

Independent perfusion of central (descending aorta) and peripheral (carotid arteries) circuits was then initiated by pulling fresh media from two different reservoirs of a custom-built tonometer. This custom-built system was designed to accommodate a common return while preventing mixing of perfusate once equilibration was achieved in the two reservoirs. Between the reservoir and the preparation, central and peripheral perfusate passed through a heat exchanger, bubble trapper, and 25 μm filter. After the initiation of independent perfusion, the central perfusate was equilibrated with 40 Torr PCO₂ in O₂, and the peripheral perfusate was equilibrated with 35 Torr PCO₂ and 100 Torr PO₂ in N₂. Of note, the decerebration transects the circle of Willis therefore preventing any arterial mixing of perfusates. Once stabilized and distinct breath-like movement was detected, the phrenic nerve was dissected free and attached to a suction electrode. The vagus nerves were dissected free and transected at the level of the clavicle, well below the nodose and vagal ganglia, and the right proximal vagus (descending from brainstem) was attached to a suction electrode. Neurograms were amplified (10000× Phrenic, 20000× Vagus; Differential AC Amplifier Model 1700, A-M Systems Inc., Carlsborg, Wash., USA), filtered (low cut-off, 300 Hz, high cut-off, 5 kHz), rectified and integrated (200-ms time constant, CWE moving averager, Ardmore, Pa.) and computer archived (Digidata 1322A and Axoscope 9.0, Axon Instruments/Molecular Devices, Union City, Calif., USA) at a sampling rate of 5 kHz, and analysed off-line with custom software written in VEE (R.J.A.W.). Once carotid body activity was assessed with a hypoxic bout (50 Torr O₂, 40 Torr CO₂) normoxic conditions were re-established; any preparation which failed to demonstrate a hypoxic response was discarded. Upon recovery from hypoxia, the brainstem was made hypocapnic, in order to render the preparation apneic. Once apnea was achieved, LPA (18:1, 5 μM) was delivered into the line supplying the carotid body. Ten minutes were given to record the presence or absence of a response, upon which normoxia was re-established to demonstrate the viability of the preparation. This experiment was completed in n=6 carotid body intact and n=6 carotid body denervated preparations. Variables were normalized to the initial normoxic condition. The difference between LPA and hypocapnic conditions was calculated for phrenic (nVT—neural tidal volume (amplitude, normalized units), fR—frequency (bursts·min⁻¹), nVE (neural minute ventilation, fRxnVT—normalized units) and vagal total activity (normalized units) and used for statistical analysis. Differences between intact and denervated preparations were compared with Mann Whitney Rank Test (fR,nVT and nVE) or Student's unpaired two-sided t-test (Vagus).

### Example 12: In Vivo Demonstration of LPA Mediated Bronchoconstriction

To investigate the carotid body-mediated broncho constricting pathway in response to LPA, naïve Brown Norway (160-200 g) rats were anesthetized with isoflurane (5%, balance O₂) and instrumented for surgery. The femoral artery and vein were cannulated for the measurement of arterial pressure, the infusion of intravenous anesthetic, alfaxan (˜15 mg/kg/min) delivered by syringe pump (Kent Scientific, Torrington Conn.) and the jugular vein was cannulated for the delivery of drugs and saline. The trachea was cannulated and the rat was subsequently paralyzed with pancuronium bromide (1 mg/kg, ia, dissolved in 0.9% saline) and the animal attached to the Flexivent respirator system (SCIREQ, Montreal QC) for ventilation and measurement of airway resistance. Upon stabilization to the ventilator and intervention, single oscillator maneuvers (Snapshot 90) were repeated 5 times during saline (control condition), injection of 5 μM LPA bolus into the jugular vein, and finally an injection of 6 μM bolus sodium cyanide (iv, an independent test of carotid body function⁴⁴) at least 10 min were given between challenges. This experiment was repeated in n=6 rats with intact carotid sinus nerves and n=6 bilateral carotid sinus nerve denervated rats.

In order to demonstrate that C-fibre mediated vagal-vagal reflexes remain intact in carotid body denervated rats, naïve Brown Norway carotid body denervated rats with vagus intact (n=6) or denervated (n=6) were exposed to aerosolized capsaicin (100 breaths, 50 μM) and lung resistance was measured at 2 and 10 min⁷³. The average of the 5 single oscillator maneuvers was taken to calculate total lung resistance (R_{L}) for each time point^{118,119} expressed as normalized units (normalized to the baseline saline condition). Data were analyzed using Student's unpaired two-sided t-test for each condition for LPA injection and NaCN. Capsaicin data were analyzed with two-way repeated measures and one-way repeated measures ANOVA.

### Example 13: Asthmatic Model

Brown Norway rats (80-120 g) were sensitized to ovalbumin (1 mg) with pertussis toxin (0.5 ng) and aluminum hydroxide as adjuvant (0.15 g) dissolved in saline (1 ml ip; OVA sensitized), or saline (1 ml ip; Naïve) for three consecutive days (Days 1, 2, and 3) and challenged with aerosolized 5% ovalbumin (OVA, dissolved in 0.9% saline) or saline (Naïve) on days 15, 18 and 21 for 10 min. All anaesthetized experiments conducted on OVA and Naïve rats were conducted ˜7 days (day 28) following the last aerosol exposure. Differences in timeline are indicated for specific experiments below.

### Example 14: Lung Mechanics and Respiratory Distress in OVA Sensitized Rats

Seven cohorts of OVA sensitized Brown Norway rats were used for certain experiments (see above).

OVA Cohort 1 (n=26): To determine the effect of bradykinin and OVA-sensitization on airway resistance (RL) OVA-sensitization (day 28) and naïve rats were anesthetized with isoflurane (5%, balance O₂) and instrumented for surgery. The femoral artery and vein were cannulated for the measurement of arterial pressure, the infusion of intravenous anesthetic, alfaxan (15 mg/kg/min delivered by syringe pump; Kent Scientific, Torrington Conn.) and the jugular vein was cannulated for the delivery of saline (and drugs in several of the cohorts described below). The trachea was cannulated and the rat was subsequently paralyzed with pancuronium bromide (1 mg/kg, ia, dissolved in 0.9% saline) and the animal attached to the Flexivent respirator system (SCIREQ, Montreal QC) for ventilation and measurement of airway mechanics. Bradykinin (0.4 mg) was nebulized for 30 breaths at 1, 10 and 20 min following an initial (saline) baseline challenge. Single oscillator maneuvers (Snapshot 90) were repeated 5 times during baseline challenge and each bradykinin inhalation and the average of the 5 maneuvers was taken to calculate total lung resistance (RL) and/or elastance (EL) for each time point^{74,75}. Responses to bradykinin was normalized to the initial saline baseline challenge. Data were analyzed with two-way repeated measures ANOVA (time×group).
OVA Cohort 2 (n=48): To investigate the carotid body-vagal-lung broncho constricting pathway, naïve and OVA-sensitized rats were instrumented and attached to the Flexivent as in Cohort 1. OVA-sensitized rats were randomly separated into 7 different groups: no intervention (OVA), vagotomy (VaG), carotid body denervation (CB), LPAr blockade (BrP-LPA; 3 mg/kg iv, dissolved in dimethyl sulfoxide), LPAr blockade (Ki16425; 5 mg/kg iv, dissolved in dimethyl sulfoxide), TRPV1 Blockade (AMG9810; 10 μM/kg iv, 10× solution dissolved in 0.6 ml tween 80 and 0.4 ml 100% ethanol and diluted in ddH₂O), LPAr+TRPV1 blockade (iv). Once surgical procedure and intervention (antagonist injection or nerve dissection) were complete, rats were allowed to stabilize for 30 min while being ventilated. Airway mechanics were measured in response to bradykinin nebulizations (as in Cohort 1). Heart rate was attained from the blood pressure waveform, SaO₂ was monitored from a pulse oximeter (Kent Scientific, Torrington Conn.) and blood gases were analyzed from 0.1 ml arterial samples drawn before and after bradykinin with a blood gas analyzer (Element POC, Heska Barrie, ON). Data were analyzed using two-way repeated measures ANOVA (group×time) and certain results are shown in FIGS. 4A and 4B.
OVA Cohort 3 (n=11): To ensure effects of carotid body denervation were persistent, the effects of bradykinin 4-5 days after carotid body denervation were tested in OVA sensitized animals. Bilateral carotid body denervation was performed under ketamine/xylazine (100 mg/kg IM) anesthetic. In sham counterparts, carotid bodies were identified but left intact. Both groups of animals received buprenorphine (0.05 mg/kg SQ) post-operatively. Airway mechanics were measured in response to bradykinin nebulizations (as in Cohort 1). Data were analyzed with two-way repeated measures ANOVA (group×time).
OVA Cohort 4 (n=12): To test if the bradykinin-mediated asthmatic bronchoconstriction involves a distinct mechanism to the C-fibre mediated vagal-vagal reflex, all animals were bilaterally carotid body denervated to eliminate the lung-carotid body-lung reflex. OVA sensitized rats were instrumented to the Flexivent (see Cohort 1) and carotid body denervated. 6 rats were also vagotomised to evaluate the contribution of the C-fibre mediated vagal-vagal pathway. Upon stabilization, the C-fibre mediated vagal-vagal pathway was activated with aerosolized capsaicin (100 breaths, 50 μM) and airway mechanics measured at 2 and 10 min¹²⁰. Data were analyzed with two-way repeated measures ANOVA (group×time).
Cohort 5 (n=12): To test if LPA has local broncho constricting effects, rats were instrumented to the Flexivent (see Cohort 1) and either bilaterally carotid body denervated or sham exposed. Upon stabilization, LPA (5 μM) was nebulized for 100 breaths and RL measured at 1 and 30 min post-inhalation¹¹⁰ as above. Data were analyzed with two-way repeated measures ANOVA (group×time).
OVA Cohort 6 (n=6): To characterize breathing in the asthmatic model, rats were studied in a plethysmograph. In this cohort, prior to sensitization, rats were exposed to OVA aerosol for 10 min and then placed in a plethysmograph (Buxco, DSI systems Minneapolis, Minn.) to obtain baseline measurements. On day 21, following their final aerosol exposure, plethysmography was repeated to determine effects of OVA sensitization. Increases in expiratory time (Te) and decreases in inspiratory time: expiratory time ratio (Ti:Te) were evaluated as indices of respiratory difficulty/airway constriction. Data were averaged in 5-min bins and compared using two-way repeated measures ANOVA (time×group) and are presented in FIG. 6a-c.
OVA Cohort 7 (n=12): To demonstrate therapeutic effectiveness of the antagonist intervention following allergen provocation, a subset of n=12 OVA rats underwent separate plethysmograph experiments. Rats were OVA-sensitized as described above, but on day 15, following OVA exposure, rats were treated with saline or vehicle; on day 18 and 21, aerosolized ovalbumin was delivered for 10 min, followed 10 min later by saline, vehicle, or LPA receptor blocking cocktail delivered ip. The LPA receptor blocking cocktail contained 3 mg/kg BrP-LPA to block LPA-specific receptors (dissolved in dimethyl sulfoxide) and 10 μM/kg AMG9810 to block TRPV1 (10× solution dissolved in 0.6 ml tween 80 and 0.4 ml 100% ethanol and diluted in ddH₂O). Rats were then placed in a plethysmograph (Buxco, DSI systems Minneapolis, Minn.; total time after beginning of aerosolization=20 min) and Ti:Te and Te were measured as above for 3 hours in order to attain the early and late-onset of asthmatic responses¹²¹. Five-minute averages of each variable were calculated. Data was analyzed using a two-way repeated measures ANOVA (time×group) and are presented in FIG. 6d-h.

### REFERENCES

¹ Global Burden of Disease Study 2013 Collaborators. Global, regional, and national incidence, prevalence, and years lived with disability for 301 acute and chronic diseases and injuries in 188 countries, 1990-2013: a systematic analysis for the Global Burden of Disease Study 2013. Lancet Lond. Eng;. 386,743-800 (2015).
² Centers for Disease Control and Prevention; National Health Statistics, see the web site www.cdc.gov/nchs/fastats/asthma.htm.
³ Wener R. R. L. & Bel E. H. (2103) Severe refractory asthma: an update, Eur. Respiratory Review, 22:227-235.
⁴ SIGN 141 British Guideline on the Management of Asthma (October 2014) available from the web site www.brit-thoracic.org.uk/document-library/clinical-information/asthma/btssign-asthma-guideline-2014/.
⁵ 2017 GINA Report: Global Strategy for Asthma Management and Prevention. Global Initiative for Asthma—GINA.
⁶ McFadden, E. R. & Warren, E. L. Observations on asthma mortality. Ann. Intern. Med. 127,142-147 (1997).
⁷ Wenzel, S. E. Asthma phenotypes: the evolution from clinical to molecular approaches. Nat Med 18,716-25 (2012).
⁸ Flood-Page, P. et al. A study to evaluate safety and efficacy of mepolizumab in patients with moderate persistent asthma. Am. J. Respir. Crit. Care Med. 176, 1062-1071 (2007).
⁹ Borish, L. C. et al. Interleukin-4 receptor in moderate atopic asthma. A phase I/II randomized, placebo-controlled trial. Am. J. Respir. Crit. Care Med. 160, 1816-1823 (1999).
¹⁰ Leckie, M. J. et al. Effects of an interleukin-5 blocking monoclonal antibody on eosinophils, airway hyper-responsiveness, and the late asthmatic response. Lancet Lond. Engl. 356, 2144-2148 (2000).
11 Jayaram, L. et al. Determining asthma treatment by monitoring sputum cell counts: effect on exacerbations. Eur. Respir. J. 27, 483-494 (2006).
¹² Moore, W. C. et al. Identification of asthma phenotypes using cluster analysis in the Severe Asthma Research Program. Am. J. Respir. Crit. Care Med. 181, 315-323 (2010).
¹³ Miranda, C., Busacker, A., Balzar, S., Trudeau, J. & Wenzel, S. E. Distinguishing severe asthma phenotypes: role of age at onset and eosinophilic inflammation. J. Allergy Clin. Immunol. 113, 101-108 (2004).
¹⁴ Haldar, P. et al. Cluster analysis and clinical asthma phenotypes. Am. J. Respir. Crit. Care Med. 178,218-224 (2008).
¹⁵ Tattersall, M. C. et al. Asthma Predicts Cardiovascular Disease EventsSignificance. Arterioscler. Thromb. Vasc. Biol. 35, 1520-1525 (2015).
¹⁶ Barnes, P. J. Neural Control of Human Airways in Health and Disease. Am. Rev. Respir. Dis. 134, 1289-1314 (1986).
¹⁷ Sato, E., Koyama, S., Nomura, H., Kubo, K. & Sekiguchi, M. Bradykinin stimulates alveolar macrophages to release neutrophil, monocyte, and eosinophil chemotactic activity. J. Immunol. Baltim. Md 1950 157,3122-3129 (1996).
¹⁸ Eric, J., Gabra, B. H. & Sirois, P. Implication of the bradykinin receptors in antigen-induced pulmonary inflammation in mice. Br. J. Pharmacol. 138,1589-1597 (2003).
¹⁹ Ehrenfeld, P. et al. Activation of kinin B1 receptors induces chemotaxis of human neutrophils. J. Leukoc. Biol. 80,117-124 (2006).
²⁰ Broadley, K. J., Blair, A. E., Kidd, E. J., Bugert, J. J. & Ford, W. R. Bradykinin-Induced Lung Inflammation and Bronchoconstriction: Role in Parainfluenze-3 Virus-Induced Inflammation and Airway Hyperreactivity. J. Pharmacol. Exp. Ther. 335, 681-692 (2010).
²¹ Reynolds, C. J., Togias, A. & Proud, D. Airway neural responses to kinins: tachyphylaxis and role of receptor subtypes. Am. J. Respir. Crit. Care Med. 159, 431-438 (1999).
²² Ellis, K. M., Cannet, C., Mazzoni, L. & Fozard, J. R. Airway hyperresponsiveness to bradykinin induced by allergen challenge in actively sensitised Brown Norway rats. Naunyn. Schmiedebergs Arch. Pharmacol. 369,166-178 (2004).
²³ Hannon, J. P., Tigani, B., Williams, I., Mazzoni, L. & Fozard, J. R. Mechanism of airway hyperresponsiveness to adenosine induced by allergen challenge in actively sensitized Brown Norway rats. Br. J. Pharmacol. 132,1509-1523 (2001).
²⁴ Christiansen, S. C., Proud, D. & Cochrane, C. G. Detection of tissue kallikrein in the bronchoalveolar lavage fluid of asthmatic subjects. J. Clin. Invest. 79,188-197 (1987).
²⁵ Undem, B. J. & Carr, M. J. The role of nerves in asthma. Curr. Allergy Asthma Rep. 2, 159-165 (2002).
²⁶ Tränkner, D., Hahne, N., Sugino, K., Hoon, M. A. & Zuker, C. Population of sensory neurons essential for asthmatic hyperreactivity of inflamed airways. Proc. Natl. Acad. Sci. 111, 11515-11520 (2014).
27 Denjean, A., Canet, E., Praud, J. P., Gaultier, C. & Bureau, M. Hypoxia-induced bronchial responsiveness in awake sheep: role of carotid chemoreceptors. Respir Physiol 83, 201-10 (1991).
²⁸ Finley, J. C. & Katz, D. M. The central organization of carotid body afferent projections to the brainstem of the rat. Brain Res. 572, 108-116 (1992).
²⁹ Nadel, J. A. & Widdicombe, J. G. Effect of changes in blood gas tensions and carotid sinus pressure on tracheal volume and total lung resistance to airflow. J. Physiol. 163, 13-33 (1962).
³⁹ Shen, M.-Y., Luo, Y.-L., Yang, C.-H., Ruan, T. & Lai, C. J. Hypersensitivity of lung vagal C fibers induced by acute intermittent hypoxia in rats: role of reactive oxygen species and TRPA1. Am. J. Physiol.-Regul. Integr. Comp. Physiol. 303, R1175-R1185 (2012).
³¹ Vidruk, E. H. Hypoxia potentiates, oxygen attenuates deflation-induced reflex tracheal constriction. J Appl Physiol 1985 59, 941-6 (1985).
³² Widdicombe, J. G. Action potentials in parasympathetic and sympathetic efferent fibres to the trachea and lungs of dogs and cats. J. Physiol. 186, 56-88 (1966).
³³ Iscoe, S. & Fisher, J. Bronchomotor Responses to Hypoxia and Hypercapnia in Decerebrate Cats. J. Appl. Physiol. 78, 117-123 (1995).
³⁴ Haxhiu, M. A. et al. Brain stem excitatory and inhibitory signaling pathways regulating bronchoconstrictive responses. J. Appl. Physiol. 98, 1961-1982 (2005).
³⁵ Mazzone, S. B. & Canning, B. J. Central nervous system control of the airways: pharmacological implications. Curr. Opin. Pharmacol. 2, 220-228 (2002).
³⁶ Ahmed, T. & Marchette, B. Hypoxia enhances nonspecific bronchial reactivity. Am Rev Respir Dis 132, 839-44 (1985).
³⁷ Xu, F., Zhuang, J., Zhou, T. & Lee, L. Y. Ovalbumin sensitization alters the ventilatory responses to chemical challenges in guinea pigs. J Appl Physiol 1985 99, 1782-8 (2005).
³⁸ Kumar, P. & Prabhakar, N. R. Peripheral chemoreceptors: function and plasticity of the carotid body. Compr Physiol 2, 141-219 (2012).
³⁹ Nurse, C. A. Neurotransmission and neuromodulation in the chemosensory carotid body. Auton Neurosci 120, 1-9 (2005).
⁴⁰ Messenger, S. A., Moreau, J. M. & Ciriello, J. Effect of chronic intermittent hypoxia on leptin and leptin receptor protein expression in the carotid body. Brain Res. 1513, 51-60 (2013).
⁴¹ Roy, A. et al. Anandamide modulates carotid sinus nerve afferent activity via TRPV1 receptors increasing responses to heat. J. Appl. Physiol. Bethesda Md 1985 112, 212-224 (2012).
⁴² Roy, A. & Wilson, R. J. A. Encyclopedia of Neuroscience 571-576 (2009).
⁴³ Ollerenshaw, S. L., Jarvis, D., Sullivan, C. E. & Woolcock, A. J. Substance P immunoreactive nerves in airways from asthmatics and nonasthmatics. Eur Respir J 4, 673-82 (1991).
⁴⁴ Denjean, A., Canet, E., Praud, J. P., Gaultier, C. & Bureau, M. Hypoxia-induced bronchial responsiveness in awake sheep: role of carotid chemoreceptors. Respir Physiol 83, 201-10 (1991).
⁴⁵ Vidruk, E. H. Hypoxia potentiates, oxygen attenuates deflation-induced reflex tracheal constriction. J Appl Physiol 1985 59, 941-6 (1985).
⁴⁶ Anderson, J. A. et al. Carotid body resection: Approved by the executive committee of the American academy of allergy and immunology. J. Allergy Clin. Immunol. 78, 273-275 (1986).
⁴⁷ Nakayama, K. Surgical removal of the carotid body for bronchial asthma. Dis. Chest 40, 595-604 (1961).
⁴⁸ Winter, B. Bilateral carotid body resection for asthma and emphysema. A new surgical approach without hypoventilation or baroreceptor dysfunction. Int Surg 57, 458-66 (1972).
⁴⁹ Winter, B. & Whipp, B. J. Immediate effects of bilateral carotid body resection on total respiratory resistance and compliance in humans. Adv. Exp. Med. Biol. 551, 15-21 (2004).
⁵⁰ Poppius, H. & Stenius, B. Changes in arterial oxygen saturation in patients with hyperreactive airways during a histamine inhalation test. Scand J Respir Dis 58, 1-4 (1977).
⁵¹ Smith, T. F. & Hudgel, D. W. Arterial oxygen desaturation during sleep in children with asthma and its relation to airway obstruction and ventilatory drive. Pediatrics 66, 746-51 (1980).
⁵² Wagner, P. D. Gas exchange in chronic pulmonary disease. Clin Physiol 5 Suppl 3, 9-17 (1985).
⁵³ Del Rio, R., Moya, E. A., Parga, M. J., Madrid, C. & Iturriaga, R. Carotid body inflammation and cardiorespiratory alterations in intermittent hypoxia. Eur. Respir. J. 39, 1492-1500 (2012).
⁵⁴ Ackland, G. L., Kazymov, V., Marina, N., Singer, M. & Gourine, A. V. Peripheral neural detection of danger-associated and pathogen-associated molecular patterns. Crit. Care Med. 41, e85-92 (2013).
⁵⁵ Schultz, H. D., Marcus, N. J. & Del Rio, R. Mechanisms of carotid body chemoreflex dysfunction during heart failure. Exp. Physiol. 100, 124-129 (2015).
⁵⁶ Covelli, H. D., Weled, B. J. & Beekman, J. F. Efficacy of continuous positive airway pressure administered by face mask. Chest 81, 147-150 (1982).
⁵⁷ Smith, R. A., Kirby, R. R., Gooding, J. M. & Civetta, J. M. Continuous positive airway pressure (CPAP) by face mask. Crit. Care Med. 8, 483-485 (1980).
⁵⁸ Busk, M. et al. Use of continuous positive airway pressure reduces airway reactivity in adults with asthma. Eur. Respir. J. 41, 317-322 (2013).
⁵⁹ D'Amato, M. et al. Nocturnal continuous positive airway pressure in severe non-apneic asthma. A pilot study. Clin. Respir. J. 8, 417-424 (2014).
⁶⁰ Esquinas, A. M. & Özyilmaz, E. Mechanical bronchodilatation for asthmatic airway: role of CPAP therapy. Tüberküloz Ve Toraks 61, 263-264 (2013).
⁶¹ Lin, H.-C. et al. Effect of nasal continuous positive airway pressure on methacholine-induced bronchoconstriction. Respir. Med. 89, 121-128 (1995).
⁶² Roberts, J. A., Raeburn, D., Rodger, I. W. & Thomson, N. C. Comparison of in vivo airway responsiveness and in vitro smooth muscle sensitivity to methacholine in man. Thorax 39, 837-843 (1984).
⁶³ Tsuji, F. & Aono, H. Role of Transient Receptor Potential Vanilloid 1 in Inflammation and Autoimmune Diseases. Pharmaceuticals 5,837-852 (2012).
⁶⁴ Cantero-Recasens G, Gonzalez J R, Fandos C, Duran-Tauleria E, Smit L a M, Kauffmann F, Antó J M, Valverde Ma. Loss of function of transient receptor potential vanilloid 1 (TRPV1) genetic variant is associated with lower risk of active childhood asthma. J Biol Chem. 2010; 285:27532-5.
⁶⁵ Ren Y-F, Li H, Xing X-H, Guan H-S, Zhang B-A, Chen C-L, Zhang J-H. Preliminary study on pathogenesis of bronchial asthma in children. Pediatr Res. 2015; 77:506-10.
⁶⁶ L. Y. Lee & Q. Gu (2009) “Role of TRPVI in inflammation-induced airway hypersensitivity,” Curr Opin Pharmacol., 9(3):243-249.
⁶⁷ Sadofsky L R, Ramachandran R, Crow C, Cowen M, Compton S J, Morice A H. Inflammatory stimuli up-regulate transient receptor potential vanilloid-1 expression in human bronchial fibroblasts. Exp Lung Res. 2012; 38:75-81.
⁶⁸ McGarvey L P, Butler C A, Stokesberry S, Polley L, McQuaid S, Abdullah H, Ashraf S, McGahon M K, Curtis T M, Arron J, Choy D, Warke T J, Bradding P, Ennis M, Zholos A, Costello R W, Heaney L G. Increased expression of bronchial epithelial transient receptor potential vanilloid 1 channels in patients with severe asthma. J Allergy Clin Immunol. 2014; 133:704-12.
⁶⁹ Watanabe N, Horie S, Spina D, Michael G J, Page C P, Priestley J V. Immunohistochemical localization of transient receptor potential vanilloid subtype 1 in the trachea of ovalbumin-sensitized Guinea pigs. Int Arch Allergy Immunol. 2008; 146:28-32.
⁷⁰ K. Baker, K. Raemdonck, B. Dekkak, R. J. Snelgrove, J. Ford, F. Shala. M. G. Belvisi, M. A. Birrell (2016) Role of the ion channel, transient receptor potential cation channel subfamily V member 1 (TRPV1), in allergic asthma. Respiratory Research 17:67
⁷¹ Nieto-Posadas, A. et al. Lysophosphatidic acid directly activates TRPV1 through a C-terminal binding site. Nat. Chem. Biol. 8, 78-85 (2012).
⁷² Pan, H.-L., Zhang, Y.-Q. & Zhao, Z.-Q. Involvement of lysophosphatidic acid in bone cancer pain by potentiation of TRPV1 via PKCϵ pathway in dorsal root ganglion neurons. Mol. Pain 6, 85 (2010).
⁷³ Park, G. Y. et al. Autotaxin production of lysophosphatidic acid mediates allergic asthmatic inflammation. Am J Respir Crit Care Med 188, 928-40 (2013).
⁷⁴ Yoder, M. et al. Bioactive lysophosphatidylcholine 16:0 and 18:0 are elevated in lungs of asthmatic subjects. Allergy Asthma Immunol. Res. 6, 61-65 (2014).
⁷⁵ Zhuge, Y. et al. Stimulated bronchial epithelial cells release bioactive lysophosphatidylcholine 16:0, 18:0, and 18:1. Allergy Asthma Immunol. Res. 6, 66-74 (2014).
⁷⁶ Eichholtz, T., Jalink, K., Fahrenfort, I. & Moolenaar, W. H. The bioactive phospholipid lysophosphatidic acid is released from activated platelets. Biochem. J. 291, 677-680 (1993).
⁷⁷ Tigyi, G. Lipids: LPA activates TRPV1—and it hurts. Nat. Chem. Biol. 8, 22-23 (2012).
⁷⁸ David Julius, David E. Clapham. Transient Receptor Potential channels: TRPV1. Last modified on 24 Apr. 2017. Accessed on 19 Jul. 2017. IUPHAR/BPS Guide to PHARMACOLOGY; obtained from the web site: guidetopharmacology.org /GRAC/ObjectDisplayForward?objectId=507.
⁷⁹ Jerold Chun, Timothy HIa, Wouter Moolenaar, Sarah Spiegel, Yun C. Yung, Chido Mpamhanga. Lysophospholipid (LPA) receptors. Accessed on 19 Jul. 2017. IUPHAR/BPS Guide to PHARMACOLOGY, obtained from web site guidetopharmacology.org/GRAC/FamilyDisplayForward?familyId=36.
⁸⁰ B. Veronesi & M. Oortgiesen (2006) The TRPV1 Receptor: Target of Toxicants and Therapeutics, Toxicological Sciences 89(1):1-13.
⁸¹ M. E. Kort & P. R. Kym (2012) TRPV1 Antagonists: Clinical Setbacks and Prospects for Future Development Prog Med Chem. 51:57-70.
⁸² Watabiki, T., Kiso, T., Kuramochi, T., Yonezawa, K., Tsuji, N., Kohara, A., Kakimoto, S., Aoki, T. and Matsuoka, N.J. (2011) J. Pharmacol. Exp. Ther. 336, 743-750.
⁸³ Reilly, R. M. et al. (2012) Pharmacology of Modality-Specific Transient Receptor Potential Vanilloid-1 Antagonists That Do Not Alter Body Temperature. J. Pharmacology and Experimental Therapeutics August 2012, 342 (2) 416-428.
⁸⁴ G. Y. Wong & N. R. Gavva (2008) Therapeutic potential of vanilloid receptor TRPV1 agonists and antagonists as analgesics: Recent advances and setbacks, Brain Research Reviews 60:267-277
⁸⁵ Choi J W, Herr D R, Noguchi K, Yung Y C, Lee C W, Mutoh T, Lin M E, Teo S T, Park K E, Mosley A N, Chun J. (2010) LPA receptors: subtypes and biological actions, Annu. Rev. Pharmacol. Toxicol, 50:157-186.
⁸⁶ Y. C. Yung, Nicole C. Stoddard, and Jerold Chun (2014) LPA receptor signaling: pharmacology, physiology, and pathophysiology, J. Lipid Research, 55:1192-12-14.
87 Beck H P, Kohn T, Rubenstein S, Hedberg C, Schwandner R, Hasslinger K, Dai K, Li C, Liang L, Wesche H, Frank B, An S, Wickramasinghe D, Jaen J, Medina J, Hungate R, Shen W, Discovery of potent LPA2 (EDG4) antagonists as potential anticancer agents. Bioorg Med Chem Lett. 2008 Feb. 1; 18(3):1037-41. ⁸⁸ Fells J I, Tsukahara R, Liu J, Tigyi G, Parrill A L (2009) Structure-based drug design identifies novel LPA3 antagonists. Bioorg Med Chem. November 1,17(21):7457-64.
⁸⁹ Cummings, K. J. & Wilson, R. J. A. Time-dependent modulation of carotid body afferent activity during and after intermittent hypoxia. Am. J. Physiol. Regul. Integr. Comp. Physiol. 288, R1571-1580 (2005).
⁹⁰ Day, T. A. & Wilson, R. J. A. Specific carotid body chemostimulation is sufficient to elicit phrenic poststimulus frequency decline in a novel in situ dual-perfused rat preparation. Am. J. Physiol. Regul. Integr. Comp. Physiol. 289, R532-R544 (2005).
⁹¹ Fiamma, M.-N., O'Connor, E. T., Roy, A., Zuna, I. & Wilson, R. J. A. The essential role of peripheral respiratory chemoreceptor inputs in maintaining breathing revealed when CO2 stimulation of central chemoreceptors is diminished. J. Physiol. 591, 1507-1521 (2013).
⁹² Bice, D. E., Seagrave, J. & Green, F. H. Animal models of asthma: potential usefulness for studying health effects of inhaled particles. Inhal. Toxicol. 12, 829-862 (2000).
⁹³ Pellegrino, R., Wilson, O., Jenouri, G. & Rodarte, J. R. Lung mechanics during induced bronchoconstriction. J. Appl. Physiol. 81, 964-975 (1996).
⁹⁴ Winter, B. Bilateral Carotid-Body Resection in Children with Severe Bronchial-Asthma—a Retrospective Study. Vasc. Surg. 15, 399-408 (1981).
⁹⁵ Whipp, B. J. Carotid bodies and breathing in humans. Thorax 49, 1081-1084 (1994).
⁹⁶ Bencini, C. & Pulera, N. The carotid bodies in bronchial asthma. Histopathology 18, 195-200 (1991).
⁹⁷ Mitzner, W. Airway Smooth Muscle. Am. J. Respir. Crit. Care Med. 169, 787-790 (2004).
⁹⁸ Widdicombe, J. Chemoreceptor Control of the Airways. Respir. Physiol. 87, 373-381 (1992).
⁹⁹ Ferrante, E., Quatela, M. M., Pistelli, R. & Fuso, L. Hypoxemia in young asthmatics with mild airway obstruction by methacholine challenge. Mil Med 164, 820-3 (1999).
¹⁰⁰ Stewart, I. C., Parker, A., Catterall, J. R., Douglas, N. J. & Flenley, D. C. Effect of bronchial challenge on breathing patterns and arterial oxygenation in stable asthma. Chest 95, 65-70 (1989).
¹⁰¹ D'Brot, J. & Ahmed, T. Hypoxia-induced enhancement of nonspecific bronchial reactivity: role of leukotrienes. J. Appl. Physiol. Bethesda Md 1985 65, 194-199 (1988).
¹⁰² Perrin, K. et al. Randomised controlled trial of high concentration versus titrated oxygen therapy in severe exacerbations of asthma. Thorax 66, 937-941 (2011).
¹⁰³ McFadden, E. R. & Lyons, H. A. Arterial-blood gas tension in asthma. N. Engl. J. Med. 278, 1027-1032 (1968).
¹⁰⁴ Saito, H. et al. Effect of mild hypoxia on airway responsiveness to methacholine in subjects with airway hyperresponsiveness. Chest 116, 1653-8 (1999).
¹⁰⁵ Georas, S. N. Allergic to autotaxin. A new role for lysophospholipase d and lysophosphatidic Acid in asthma? Am J Respir Crit Care Med 188, 889-91 (2013).
¹⁰⁶ Knowlden, S. & Georas, S. N. The autotaxin-LPA axis emerges as a novel regulator of lymphocyte homing and inflammation. J Immunol 192, 851-7 (2014).
¹⁰⁷ Duff, R. F. et al. Semi-Quantitative Analysis of Plasma Lysophosphatidic Acid Levels in Patients With and Without Asthma. Am. J. Respir. Crit. Care Med. 181, (2010).
¹⁰⁸ Zhao, Y. & Natarajan, V. Lysophosphatidic acid (LPA) and its receptors: role in airway inflammation and remodeling. Biochim Biophys Acta 1831, 86-92 (2013).
¹⁰⁹ Toews, M. L., Ustinova, E. E. & Schultz, H. D. Lysophosphatidic acid enhances contractility of isolated airway smooth muscle. J. Appl. Physiol. Bethesda Md 1985 83, 1216-1222 (1997).
¹¹⁰ Hashimoto, T., Nakano, Y., Ohata, H. & Momose, K. Lysophosphatidic acid enhances airway response to acetylcholine in guinea pigs. Life Sci 70, 199-205 (2001).
¹¹¹ Fan, J. et al. Interleukin-6 increases intracellular Ca2+ concentration and induces catecholamine secretion in rat carotid body glomus cells. J. Neurosci. Res. 87, 2757-2762 (2009).
¹¹² Liu, X., He, L., Stensaas, L., Dinger, B. & Fidone, S. Adaptation to chronic hypoxia involves immune cell invasion and increased expression of inflammatory cytokines in rat carotid body. Am. J. Physiol.—Lung Cell. Mol. Physiol. 296, L158-L166 (2009).
¹¹³ Del Rio, R., Moya, E. A. & Iturriaga, R. Contribution of inflammation on carotid body chemosensory potentiation induced by intermittent hypoxia. Adv. Exp. Med. Biol. 758, 199-205 (2012).
¹¹⁴ Schuster, M., Tschernig, T., Krug, N. & Pabst, R. Lymphocytes migrate from the blood into the bronchoalveolar lavage and lung parenchyma in the asthma model of the brown Norway rat. Am. J. Respir. Crit. Care Med. 161, 558-566 (2000).
¹¹⁵ Schneider, T., van Velzen, D., Moqbel, R. & Issekutz, A. C. Kinetics and quantitation of eosinophil and neutrophil recruitment to allergic lung inflammation in a brown Norway rat model. Am. J. Respir. Cell Mol. Biol. 17, 702-712 (1997).
¹¹⁶ Ellis, R., Leigh, R., Southam, D., O'Byrne, P. M. & Inman, M. D. Morphometric analysis of mouse airways after chronic allergen challenge. Lab. Investig. J. Tech. Methods Pathol. 83, 1285-1291 (2003).
¹¹⁷ Livak, K. J. & Schmittgen, T. D. Analysis of relative gene expression data using real-time quantitative PCR and the 2(-Delta Delta C(T)) Method. Methods San Diego Calif 25, 402-408 (2001).
¹¹⁸ Davis, G. M., Coates, A. L., Dalle, D. & Bureau, M. A. Measurement of pulmonary mechanics in the newborn lamb: a comparison of three techniques. J. Appl. Physiol. Bethesda Md 1985 64, 972-981 (1988).
¹¹⁹ Hirota, J. A., Ellis, R. & Inman, M. D. Regional differences in the pattern of airway remodeling following chronic allergen exposure in mice. Respir. Res. 7, 120 (2006).
¹²⁰ Jiao, H. et al. Therapeutic effects of naringin in a guinea pig model of ovalbumin-induced cough-variant asthma. Pulm. Pharmacol. Ther. 33, 59-65 (2015).
¹²¹ Raemdonck, K. et al. A role for sensory nerves in the late asthmatic response. Thorax 67, 19-25 (2012).

1. A method for treating asthma or preventing an asthma attack which comprises administering an effective amount or a combined effective amount of one or more TRPV1 antagonist, one or more LPAr antagonist or a combination of one or more TRPV1 antagonist and one or more LPAr antagonist to a patient in need of such treatment.

2. The method of claim 1, wherein treating asthma is treating an asthma attack; or wherein treating asthma is treating acute asthma; or wherein treating asthma is treating severe acute asthma; or wherein treating asthma is treating refractory asthma.

3-5. (canceled)

6. The method of claim 1 for preventing an asthma attack.

7. The method of claim 1, wherein a combination of one or more TRPV1 antagonist and one or more LPAR antagonist is administered.

8. The method of claim 7, wherein the antagonists are formulated together or are formulated separately, but administered within 24 hours of each other.

9-11. (canceled)

12. The method of claim 1, wherein administration is by inhalation, or injection.

13-15. (canceled)

16. The method of claim 1, wherein the one or more TRPV1 antagonist are administered by a different route than the one or more LPAr antagonist.

17. The method of claim 1, wherein the one or more TRPV1 antagonist are administered by the same route as the one or more LPAr antagonist.

18. (canceled)

19. A pharmaceutical composition comprising one or more TRPV1 antagonist, one or more LPAr antagonist or a combination of one or more TRPV1 antagonist and one or more LPAr antagonist and optionally a pharmaceutically acceptable carrier for use in the treatment of asthma, or in the prevention of asthma attacks.

20. (canceled)

21. The pharmaceutical composition of claim 19, which comprises a combination of one or more TRPV1 antagonist and one or more LPAr antagonist.

22. (canceled)

23. The pharmaceutical composition of claim 19, which comprises a combination of one or more TRPV1 antagonist and one or more LPAr antagonist and wherein the molar ratio of TRPV1 antagonist to LPAr antagonist in the composition ranges from 50:1 to 1:50.

24. The pharmaceutical composition of claim 19, which comprises a combination of one or more TRPV1 antagonist and one or more LPAr antagonist and wherein the molar ratio of TRPV1 antagonist to LPAr antagonist in the composition ranges from 10:1 to 1:10.

25. A kit for treating asthma which comprises one or more TRPV1 antagonist and one or more LPAr antagonist separately packaged for use together.

26. The kit of claim 25, wherein the one or more TRPV1 antagonist and one or more LPAr antagonist are separately formulated for administration at the same time.

27. A kit for preventing an asthma attack which comprises one or more TRPV1 antagonist and one or more LPAr antagonist separately packaged for use together.

28. The kit of claim 27, wherein the one or more TRPV1 antagonist and one or more LPAr antagonist are separately formulated for administration at the same time.

29-38. (canceled)
